(19) United States
(12) Patent Application Publication
(10) Pub. No.: US 2009/0105341 A1
(43) Pub. Date: Apr. 23, 2009

(54) PROCESS FOR THE PRODUCTION OF TRANS-10, CIS 12 OCTADECADIENOIC ACID

(75) Inventor: Catherine Stanton, Cork (IE)

Correspondence Address: CONNOLLY BOVE LODGE & HUTZ, LLP, P O BOX 2207, WILMINGTON, DE, 19899, US

(73) Assignee: Teagasc Dairy Products Research Centre, Cork (IE)

(21) Appl. No.: 12/158,808

(22) PCT Filed: Nov. 29, 2006
(86) PCT No.: PCT/EP06/69030

§ 371 Date: Jun. 23, 2008

(30) Foreign Application Priority Data
Dec. 24, 2005 (EP) 05113089.6

(51) Int. Cl. A61K 31/201; C12P 7/64; C12N 1/00; A23K 1/16; A61P 35/00; C07C 57/03

(52) U.S. Cl. 514/560; 435/134; 435/243; 554/224; 426/611; 426/61

(57) ABSTRACT

The present application is directed to a process for the production of trans-10, cis-12 conjugated linoleic acid in a transgenic microorganism comprising the steps of: (a) introducing into said microorganism at least one nucleic acid molecule encoding a trans-10, cis-12 conjugated linoleic acid isomerase, (b) culturing the transgenic microorganism obtained under (a), (c) inducing the production of trans-10, cis-12 conjugated linoleic acid by adding linoleic acid to the culture, (d) incubating the induced culture for at least 12 hours, and (e) isolating the conjugated linoleic acid from the culture media and/or transgenic microorganism.

### FIELD OF THE INVENTION

The present invention relates to a process for the production of trans-10, cis 12 octadecadienoic acid, by the aid of transgenic microorganism expressing a nucleic acid molecule encoding a trans-10, cis-12 conjugated linoleic acid isomerase. The invention furthermore relates to a process for the production of feed or food products enriched in conjugated linoleic acid, in particular nutraceuticals.

The present invention also relates to feed-, food-products and nutraceuticals enriched in conjugated linoleic acid and to transgenic microorganisms expressing an alien gene encoding a trans-10, cis-12 conjugated linoleic acid isomerase and to the use of the same as probiotics in food or feed. An additional embodiment of the current invention relates to the fermented oil produced according to the inventive method and the use of said fermented oil for the production of medicaments.

### BACKGROUND OF THE INVENTION

Fatty acids and triglycerides have a multiplicity of applications in the food industry, animal nutrition, cosmetics and in the pharmaceutical sector. Depending on whether they are free saturated or unsaturated fatty acids or triglycerides with an increased content of saturated or unsaturated fatty acids, they are suitable for a very wide range of applications; thus, for example, polyunsaturated fatty acids are added to baby formula to increase the nutritional value. The various fatty acids and triglycerides are obtained mainly from microorganisms such as Mortierella or from oil-producing plants such as soya, oilseed rape, sunflowers and others, where they are usually obtained in the form of their triacyl glycerides. Alternatively, they are obtained advantageously from animals, such as fish. The free fatty acids are prepared advantageously by hydrolysis.

Whether oils with unsaturated or with saturated fatty acids are preferred depends on the intended purpose; thus, for example, lipids with unsaturated fatty acids, specifically polyunsaturated fatty acids, are preferred in human nutrition since they have a positive effect on the cholesterol level in the blood and thus on the possibility of heart disease. They are used in a variety of dietetic foodstuffs or medicaments.

Especially valuable and sought-after unsaturated fatty acids are the so-called conjugated unsaturated fatty acids, such as conjugated linoleic acid. A series of positive effects have been found for conjugated fatty acids; thus, the administration of conjugated linoleic acid reduces body fat in humans and animals, and increases the conversion of feed into body weight in the case of animals (WO 94/16690, WO 96/06605, WO 97/46230, WO 97/46118). By administering conjugated linoleic acid, it is also possible to positively affect, for example, allergies (WO 97/32008) or cancer (Banni et al., Carcinogenesis, Vol. 20, 1999: 1019-1024, Thompson et al., Cancer, Res., Vol. 57, 1997: 5067-5072).

Conjugated linoleic acid (CLA) comprises a family of positional and geometric isomers of linoleic acid (LA) with two conjugated double bonds. Most biological activity has been reported for the cis-9, trans-11 CLA (c9, t11 CLA) and the trans-10, cis-12 CLA (t10, c12 CLA) isomers including anticarcinogenic, antiatherosclerotic, antidiabetogenic, antiobesity, immune enhancing responses and positive effects on bone formation (Belury, 2002; Pariza et al., 1999; Pariza et al., 2000). Many recent studies have shown that specifically the t10, c12 CLA isomer has the ability to alter body composition by reducing the body fat content and increasing the lean body tissue in both animals and humans. Rodent feeding studies with the t10, c12 CLA isomer were associated with reduced body fat, enhanced body water, enhanced body protein and enhanced body ash (Park et al, 1999; de Deckere et al., 1999). In mouse tissue culture, the t10, c12 CLA isomer reduced lipoprotein lipase activity and intracellular triglyceride concentrations (Park et al. 1999). Other mouse studies have shown this isomer to decrease the expression of hepatic stearoyl-CoA desaturase mRNA (Lee et al, 1998) and the expression of stearoyl-CoA desaturase activity in mouse adipocytes (Choi et al, 2000), which can depress fat synthesis. Furthermore, Ostrowski et al. (1999) demonstrated ingested conjugated linoleic acid (mixture of isomers containing ˜30% t10, c12 CLA) led to increased lean tissue and decreased fat deposition in growing pigs, and Brown et al. (2003) revealed that the t10, c12 CLA isomer specifically down regulates triglyceride accumulation and PPARgamma expression in human pre-adipocytes as well as mature adipocytes. Human CLA supplementation to a group of 53 healthy men and women (4.2 g/d; equal amounts c9, t11 and t10, c12 CLA) reduced the proportion of body fat by 3.8% compared with the control group given olive oil (Smedman and Vessby, 2001). Similar results were observed by Blankson et al., 2000, who reported doses of >3.4 g CLA/d (equal amounts c9, t11 and t10, c12 CLA) to significantly reduce body fat mass in overweight and obese humans after 12 weeks treatment compared to the control group. Similarly, Thom et al. (2001) showed comparable results after a 12 weeks trial. The t10, c12 CLA is also the isomer responsible for reduction of milk fat synthesis in dairy cows. A 4 days abomasal infusion of the t10, c12 CLA isomer caused a 42% reduction in milk fat percentage and a 44% decrease in milk fat yield, whereas the c9, t11 CLA had no effect on milk fat (Baumgard et al., 2000). The mechanisms by which the t10, c12 CLA isomer inhibits milk fat synthesis are unknown but could include inhibiting of activity or synthesis of key enzymes involved in de novo fatty acid synthesis such as acetyl-CoA carboxylase and fatty acid synthetase (Baumgard et al. 2000). Therefore the evidence suggest that CLA plays an important role in health promotion and that specifically the t10, c12 CLA isomer may be useful in treatment of overweight and obese animal and human subjects. By enrichment of this isomer and incorporation into functional foods and thus make it available on a daily basis, it may have a large potential in prevention and treatment of these conditions.

Both the t10, c12 and the c9, t11 CLA isomers have been reported to exert anti-carcinogenic activity. In particular, it has been shown to inhibit skin tumor initiation and forestomach neoplasia as well as inhibiting chemically induced skin tumor promotion and mammary and colon tumorigenesis (Belury, 2002). The mechanisms by which CLA exerts the many physiological effects is not yet fully understood, but at least two different models have been proposed. One model suggests that CLA reduces the arachidonate pool leading to a reduced production of downstream eicosanoid products, which modulates cytokine production involved in inflammation and cancer. The other model includes regulation of expression of genes known to control lipid oxidation, adipocyte differentiation, energy balance and atherogenesis (Beluri, 2002; Pariza et al, 2000).

CLA can be manufactured synthetically from alkaline isomerization of linoleic and linolenic acids, or vegetable oils containing linoleic or linolenic acids. Two reactions are catalyzed when heating oil at 180° C. under alkaline conditions; hydrolysis of the fatty acid ester bond from the triglyceride lipid backbone, which produces free fatty acids, and conjugation of unconjugated unsaturated fatty acids with two or more appropriate double bonds (WO 99/32604). This method produces about 20-35% cis-9, trans-11 CLA and about the same amount of trans-10, cis-12 CLA, but enrichment of either of the isomers relative to the other is possible by using a fractional crystallization procedure. In addition, other isomers are produced mainly trans, trans isomers.

The chemical preparation of conjugated fatty acids, for example conjugated linoleic acid, is also described in U.S. Pat. No. 3,356,699 and U.S. Pat. No. 4,164,505.

CLA is formed naturally as an intermediate during biohydrogenation of linoleic acid by rumen bacteria, and natural sources of CLA are consequently milk and fats from ruminants. The main CLA isomer in milk fat is the c9, t11 CLA, which accounts for 80-90% of total milk fat CLA, whereas the t10, c12 CLA isomer is only present at about 1% (Jensen, 2002). A range of cultures with ability to convert linoleic acid into the c9, t11 CLA isomer are known, in addition to the rumen microflora. It has been shown that some strains of bifidobacteria can produce CLA, mainly the cis-9, trans-11 isomer (Coakley et al, 2003; Rosberg-Cody et al, 2004). Other species reported to biosynthesise CLA isomers, mainly the c9, t11 CLA isomer are propionibacteria used as dairy starter cultures (Jiang et al. 1998), strains of the intestinal flora of rats (Chin et al., 1994) and some Lactobacillus spp (Lin et al, 1999). A number of strains of bifidobacteria were positively identified as being capable of CLA biosynthesis from free linoleic acid as a substrate by Nordgren (1999). WO 99/29886 describes the use of bacterial strains found among food grade bacteria, particularly among dairy starter cultures, which have the ability to produce CLA in vitro by fermentation.

However, only a small number of bacteria strains can be used for biotechnological CLA production and these strains can only be identified by large and laborius screening procedures. This is due to the fact that most of the available bacteria strains (i) are not able to produce CLA from free linoleic acid and/or (ii) the growth rate of these strains is drastically inhibited by free linoleic acid in the media. The patent application WO 99/29886 discloses that only 4 out of 22 tested bacteria strains were able to produce CLA from free linoleic acid and that the growth rate of 19 of the tested strains was inhibited for more than 50% by free linoleic acid in the media. Unfortunately, the four bacteria strains found to be able to produce CLA from free linoleic acid were sensitive to linoleic acid in the media. Furthermore, 70-90% of the CLA produced by the identified bacteria strains was found to be represented by the c9, t11/t9, c11-18:2 isomers, the trans-10, cis 12 octadecadienoic acid was not detected at all. The only known species able to produce t10, C12 CLA are Propionibacterium acnes (Verhulst et al., 1987) and the rumen bacteria Megasphera elsdenii YJ-4 (Kim et. al 2000)

These results demonstrate that there is still a need for the identification of bacteria strains or processes for the biotechnological production of CLA, particularly trans-10, cis 12 octadecadienoic acid, at a economically attractive level.

WO 99/32604 describes a linoleate isomerase from Lactobacillus reuteri. The enzyme activity leads to the conversion of linoleic acid to six different CLA species which are as follows: (cis,trans)-9,11-CLA, (trans,cis)-10,12-CLA, (cis,cis)-9,11-CLA, (cis,cis)-10,12-CLA, (trans,trans)-9,11-CLA and (trans,trans)-10,12-CLA.

The disadvantages of using the above-mentioned isomerase is that the yield of the reaction is very low, the purity of the CLA produced is for an industrial process not sufficient and the process takes place with only low space-time yields. This leads to economically unattractive processes.

Thus, there is still a great need for a single, economic biotechnological process for the production of CLA which does not have the above-mentioned disadvantages.

It was therefore an objective of the present invention, to provide an efficient method for the production of conjugated linoleic acid, particularly trans-10, cis 12 octadecadienoic acid in microorganisms. It was furthermore an objective of the current invention to identity microorganisms which can be used for the efficient fermentative production of conjugated linoleic acid.

We have found that the described objectives are achieved by the use of transgenic micororganisms belonging to the family of Lactobacillaceae, Streptococcaceae, Propionibacteriaceae, Enterobacteriaceae or Bifidobacteriaceae expressing a nucleic acid molecule encoding a CLA isomerase, particularly a trans-10, cis-12 conjugated linoleic acid isomerase.

It was an unexpected result, that the above mentioned organisms, when expressing a nucleic acid molecule endocing a (trans-10, cis-12) conjugated linoleic acid isomerase, are able to produce conjugated linoleic acid, particularly trans-10, cis 12 octadecadienoic acid, from linoleic acid, since growth of the wildtype cells of most of the above-mentioned microorganisms is inhibited by free linoleic acid in the media. Consequently, the skilled person would a priori not expect that these organism can be employed in fermentation processes for the production of conjugated linoleic acid. It was even more surprisingly, that expression of the trans-10, cis-12 conjugated linoleic acid isomerase enabled the transgenic organisms to convert 50% of the added linoleic acid into trans-10, cis-12 conjugated linoleic acid when expressed in Lactococcus lactis, followed by 40% and 30% conversion rates by E. coli and Lactobacillus paracasei, respectively.

### SUMMARY OF THE INVENTION

A first subject matter of the invention therefore relates to a process for the production of trans-10, cis-12 conjugated linoleic acid in a transgenic microorganism comprising the steps of:
(a) introducing into said microorganism at least one nucleic acid molecule encoding a trans-10, cis-12 conjugated linoleic acid isomerase,
(b) culturing the transgenic microorganism obtained under (a),
(c) inducing the production of trans-10, cis-12 conjugated linoleic acid by adding linoleic acid to the culture,
(d) incubating the induced culture for at least 12 hours, and
(e) isolating the conjugated linoleic acid from the culture media and/or microorganism.

In a preferred embodiment said trans-10, cis-12 conjugated linoleic acid isomerase is characterized by a sequence
i. as described by SEQ ID No. 1, or
ii. having at least 50 consecutive base pairs of the sequence described by SEQ ID No.1, or
iii. having an identity of at least 80% over a sequence of at least 100 consecutive nucleic acid base pairs to the sequence described by SEQ ID No. 1, or
iv. hybridizing under high stringent conditions with a nucleic acid fragment of at least 50 consecutive base pairs of a nucleic acid molecule described by SEQ ID No.1, or
v. encoding a polypeptide having at least 75% identity to the amino acid sequence as shown in SEQ ID No. 2 and encoding a trans-10, cis-12 conjugated linoleic acid isomerase.

In a particularly preferred embodiment said trans-10, cis-12 conjugated linoleic acid isomerase is isolated from a rumen bacteria, preferably from Megashera elsdenii YJ-4.

Additionally, the invention relates to the above described process, wherein the nucleic acid molecule encoding said trans-10, cis-12 conjugated linoleic acid isomerase is isolated from a microorganism belonging to the genus Propionibacterium, preferably from Propionibacterium acnes.

In a preferred embodiment the invention relates to a process for the production of conjugated linoleic acid in a transgenic microorganism according to the above described steps (a) to (e), characterized in that the microorganism used under (a) belong to the family selected from the group consisting of Lactobacillaceae, Streptococcaceae, Propionibacteriaceae, Enterobacteriaceae and Bifidobacteriaceae, preferably the used microorganism belong to the genus selected from the group consisting of Lactococcus, Lactobacillus, Propionibacterium, Escherichia and Bifidobacterium, more preferably said microorganism is selected from group consisting of Lactococcus lactis, Lactobacillus paracasei and Escherichia coli.

In a preferred embodiment of the invention the process for the production of conjugated linoleic acid in a transgenic microorganism according to the above described steps (a) to (e), is characterized in that the linoleic acid is added to a microorganism culture having an optical density (OD₆₀₀) of at least 0.1.

In a particularly preferred embodiment the invention relates to a process for the production of conjugated linoleic acid in a transgenic microorganism according to the above described steps (a) to (e), characterized in that the bioconversion rate of linoleic acid is higher than 10%.

Furthermore, the invention relates to a process for the production of feed or food products or nutraceuticals enriched in conjugated linoleic acid, wherein the used conjugated linoleic acid is produced according to the above described process.

The invention relates furthermore to feed-, food-products and nutraceuticals enriched in conjugated linoleic acid, wherein the conjugated linoleic acid is produced according to the above described process.

Additionally, the invention relates to transgenic microorganisms expressing a nucleic acid molecule encoding a trans-10, cis-12 conjugated linoleic acid isomerase characterized by a sequence (i) as described by SEQ ID No. 1, or (ii) having at least 50 consecutive base pairs of the sequence described by SEQ ID No.1, or (iii) having an identity of at least 80% over a sequence of at least 100 consecutive nucleic acid base pairs to the sequence described by SEQ ID No. 1, or (iv) hybridizing under high stringent conditions with a nucleic acid fragment of at least 50 consecutive base pairs of a nucleic acid molecule described by SEQ ID No. 1, or (v) encoding a polypeptide having at least 75% identity to the amino acid sequence as shown in SEQ ID No. 2, wherein said nucleic acid sequence is preferably isolated from a rumen bacteria, more preferably from Megashera elsdenii, most preferably from Megashera elsdenii YJ-4, or from a microorganism belonging to the genus Propionibacterium, preferably Propionibacterium acnes, wherein said nucleic acid molecule is functionally linked to at least one heterologous promoter sequence.

In a furthermore preferred embodiment the present invention relates to the use of the inventive transgenic microorganism, preferably microorganism belonging to the genus selected from the group consisting of Lactococcus, Lactobacillus, Propionibacterium, Escherichia and Bifidobacterium, more preferably microorganism selected from the group consisting of Bifidobacterium breve, Bifidobacterium dentium and Bifidobacterium pseudocatenulatum as probiotics in food and feed.

Additionally, the invention relates to fermented oil produced in a transgenic microorganism according to the above described inventive process.

The invention relates furthermore to the use of the fermented oil produced according to the above described inventive method for the production of a medicament for the treatment of cancer.

### BRIEF DESCRIPTION OF THE DRAWINGS

FIG. 1: The pNZ44-coPAI construct (SEQ ID No. 5).

FIG. 2: GLC chromatogram of supernatant (A) and membranes (B) following 72 h incubation with L. lactis pNZ44-coPAI and L. lactis pNZ44 (C) with 0.2 mg/ml linoleic acid. (D) GLC chromatogram of t10, c12 CLA standard. (E) GLC chromatogram of supernatant following incubation with Lb. paracasei NFBC 338 carrying the construct pMSP3535-coPAI (uninduced) and pMSP3535 (F) incubated with 0.5 mg/ml linoleic acid for 48 h. (G) GLC chromatogram of supernatant following incubation with E. coli pNZ44-coPAI and pNZ44 (H) in 0.5 mg/ml LA for 72 h. Peak 1=Linoleic Acid, peak 2=t10, c12 CLA.

FIG. 3: CLA production vs linoleic acid (LA) usage and accumulation of the fatty acids in the membranes by L. lactis pNZ44-coPAI incubated with 0.2 mg/ml LA for 72 hours. Culture was incubated in LA at OD₆₀₀=0.5.

FIG. 4: CLA production vs linoleic acid (LA) usage and accumulation of the fatty acids in the membranes by E. coli pNZ44-coPAI incubated with 0.5 mg/ml LA for 72 hours.

FIG. 5: Cell viability for SW480 cells treated with 5-25 μg fermented oils/fatty acids/ml media after 5 days incubation. Data represents cell viability expressed as percentage of ethanol control, which was set to be 100%. (A) GLC profile of LA control oil extracted from LB media following 72 hours incubation in 37° C. and cell viability (B) of SW480 following treatment with LA control oil. (C) GLC profile of GM17 media following 72 hour growth of L. lactis pNZ44-coPAI in 0.5 mg/ml LA and cell viability (D) following treatment with L. lactis t10, c12 CLA (fermented oil). (E) GLC profile of LB media following growth of E. coli pNZ44-coPAI in 0.5 mg/ml LA and cell viability (F) following treatment with E. coli t10, c12 CLA (fermented oil). (G) Cell viability following treatment with the pure synthetic t10, c12 CLA isomer (Matreya) and (H) linoleic acid (Sigma). **** Denotes values that are significantly different (p<0.001), *** denotes values that are significantly different (p<0.01), ** Denotes values that are significantly different (p<0.05), * Denotes values that are significantly different (p<0.1) compared with control oil (unfermented linoleic acid).

FIG. 6: Microscopic examination of human colon cancer cells SW480 following 5 days incubation with different oils/fatty acids. (A) Linoleic acid unfermented control oil, 5 μg/ml media (100× magnification) and (B) 25 μg/ml media (200×). (C) E. coli t10, c12 CLA (fermented oil), 5 μg/ml media (100×) and (D) 20 μg/ml media (200×). (E) L. lactis t10, c12 CLA (fermented oil), 5 μg/ml media (100×) and (F) 20 μg/ml media (200×).

### GENERAL DEFINITIONS

It is to be understood that this invention is not limited to the particular methodology, protocols, cell lines, bacteria species or genera, constructs, and reagents described as such. It must be noted that as used herein and in the appended claims, the singular forms “a” and “the” include plural reference unless the context clearly dictates otherwise. Thus, for example, reference to “a vector” is a reference to one or more vectors and includes equivalents thereof known to those skilled in the art.

About: the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list.

Animal: as used herein refers to an organism taxonomically assigned to the animal kingdom (animalia). Those which are preferred are the vertebrates (vertebrata) with the orders of the tetrapoda (land vertebrates) and fish (pisces). Particular preference is given to the classes aves (birds) and mammalia (mammals), modern humans (Homo sapiens) being comprised as a particularly preferred mammal. Very particular preference is given to the families of the True Pigs (Suidae), cattle (Bovinae), pheasants and relatives (Phasianidae), ducks, geese and swans (Anatidae), horses (Equidae), carp family (Cyprinidae) and trout family (Salmonidae). From these families the most preferred are what are termed domestic animals and farm animals. Domestic animals in the meaning of the present invention are taken to mean animals which are not free-living, are habituated to humans, and are predominantly kept by humans in the domestic residence. Particularly preferred domestic animals are cats and dogs. Farm animals in the meaning of the present invention are taken to mean animals which are kept by humans for economic purposes. Particularly preferred farm animals are the genera domestic cattle (Bos taurus), domestic chicken (Gallus gallus domesticus), domestic pig, domestic sheep (Ovis ammon aries) and domesticated types of the gray goose (Anser anser).

The term bioconversion rate: as used herein in reference to the production of conjugated linoleic acid, preferably trans-10, cis-12 conjugated linoleic acid refers to the amount of free linoleic acid given in percent that has been converted to conjugated linoleic acid after a certain fermentation period or at the end of the fermentation process. For example, to a culture of transgenic L. lactis cells expressing a trans-10, cis-12 conjugated linoleic acid isomerase 0.5 mg/ml linoleic acid was added and incubation continued for 72 hours, followed by extraction of fatty acids from samples taken from the broth. The ratio of CLA/LA in the samples is determined using GLC (gas liquid chromatography). If the ratio of CLA/LA in the samples is 1:1, the bioconversion rate is 50%.

Cell: refers to a single cell. The term “cells” refer to a population of cells. The population may be a pure population comprising one cell type. Likewise, the population may comprise more than one cell type. In the present invention, there is no limit on the number of cell types that a cell population may comprise. The cells may be synchronized or not synchronized, preferably the cells are synchronized.

Coding region or coding sequence (CDS): when used in reference to a gene refers to the nucleotide sequences which encode the amino acids found in the nascent polypeptide as a result of translation of a mRNA molecule. The coding region is bounded, in eucaryotes, on the 5′-side by the nucleotide triplet “ATG” which encodes the initiator methionine and on the 3′-side by one of the three triplets, which specify stop codons (i.e., TAA, TAG, TGA).

Conjugated linoleic acid (CLA): refers to a mixture of positional and geometric isomers of linoleic acid, involving double bonds at positions 7 and 9, 9 and 11, 10 and 12 or 11 and 13. The isomers cis-9, trans-11 and trans-10, cis-12 isomers are of particular interest, because many beneficial effects have been attributed to said isomers. The isomers can differ positionally (mainly at positions 7 and 9, 9 and 11, or 10 and 12) (Ha et al., Anticarcinogens from fried ground beef: heat-altered derivatives of linoleic acid. Carcinogenesis. 1987 December; 8(12):1881-7) and geometrically (cis-cis, cis-trans, trans-cis, trans-trans). Of the individual isomers of CLA, cis-9, trans-11-octadecadienoic acid has been implicated as the most biologically active because it is the predominant isomer incorporated into the phospholipids of cell membranes, liver phospholipids and triglycerides (Kramer et al., Distributions of conjugated linoleic acid (CLA) isomers in tissue lipid classes of pigs fed a commercial CLA mixture determined by gas chromatography and silver ion-high-performance liquid chromatography. Lipids. 1998 June; 33(6):549-58.). This is the only isomer incorporated into the phospholipid fraction of cell membranes of animals fed a mixture of CLA isomers (Ha et al., Inhibition of benzo(a)pyrene-induced neoplasia by conjugated dienoic derivatives of linoleic acid. Cancer Res. 50:1097-1101 (1990); Ip et al., Mammary cancer prevention by conjugated dienoic derivatives of linoleic acid. Cancer Res. 51:6118-6124 (1991)). This isomer is also the predominant dietary form of CLA, obtained from fats derived from ruminant animals, including milk, dairy products and meat (Chin et al., Dietary sources of conjugated dienoic isomeres of linoleic acid, a newly recognized class of anticarcinogens. J. Food Comp. and Anal. 5: 185-197 (1992)). The terms trans-10, cis-12 octadecadienoic acid and trans-10, cis-12 CLA are used herein interchangeably.

Conjugated linoleic acid isomerase (CLA): is a protein catalizing the isomerization of linoleic acid or conjugated linoleic acid isomers, characterized in that a double bond at one carbon position is transferred to another carbon position forming one of the possible CLA isomers.

Trans-10, cis-12 conjugated linoleic acid isomerase: as used in the context of this invention means an enzyme catalysing the isomerisation of linoleic acid to trans-10, cis-12 octadecadienoic acid. The terms trans-10, cis-12 octadecadienoic acid isomerase and trans-10, cis-12 CLA isomerase are used herein interchangeably.

culturing: with regard to the inventive method refers to the growth of microorganism in liquid culture under controlled conditions. Depending on the organisms used in the processes the growth conditions can be very different and are in general known to those skilled in the art. As a rule, microorganism are grown in a liquid medium which contains a carbon source, usually in the form of sugars, a nitrogen source, usually in the form of organic nitrogen sources such as yeast extract or salts such as ammonium sulfate, a phosphate source such as potassium hydrogen phosphate, trace elements such as iron salts, manganese salts, magnesium salts and, if required, vitamins, at temperatures between 0° C. and 100° C., preferably between 10° C. and 65° C., 15° C. and 55° C., more preferably between 20° C. and 50° C., 25° C. and 45° C., particularly preferred between 30° C. and 40° C. while gassing in oxygen. The organism can be grown under aerobic or anaerobic conditions. The pH of the liquid medium can be maintained at a fixed value, i.e. the pH is regulated while culture takes place. The pH should then be in a range between pH 2 and pH 9, preferably between 4 and 8.5, 4.5 and 8, more preferably between 5 and 7.5, 5.5 and 7. However, the microorganisms may also be cultured without pH regulation. Culturing can be effected by the batch method, the semi-batch method or continuously Nutrients may be supplied at the beginning of the fermentation or fed in semicontinuously or continuously. Such methods can be found in e.g Scardovi V (1986) Genus Bifidobacterium and Genus Lactobacillus. In Bergey's Manual of Systematic Bacteriology [N M P H A Sneath, M E Sharpe, J G Holt, editor]. Baltimore: Williams & Wilkins.

Expression: refers to the biosynthesis of a gene product. For example, in the case of a structural gene, expression involves transcription of the structural gene into mRNA and—optionally—the subsequent translation of mRNA into one or more polypeptides.

The term fermented oil: as used herein refers to the oil and fatty acid containing fraction produced by a microorganism during a fermentation. Fermentation is used to refer to the bulk growth of microorganisms on a growth medium. No distinction is made between aerobic and anaerobic metabolism when the word is used in the context of the present invention. The term fermented oil refers to the fatty acids fraction that can be recovered/isolated (e.g. see example 8) from the microorganism, particularly the cell membranes of the microorganism or the fermentation broth.

Functional equivalents: with regard to the invention nucleic acid sequence has to be understood as natural or artificial mutations of the SEQ ID No. 1. Mutations can be insertions, deletions or substitutions of one or more nucleic acids that do not diminish the Linoleic acid isomeration activity of the expression product of said sequence. These functional equivalents having a identity of at least 80%, preferably 85%, more preferably 90%, most preferably more than 95%, very especially preferably at least 98% identity—but less then 100% identity to the sequence as described by the SEQ ID No. 1, wherein said identity is determined over a sequence of at least 100 consecutive base pairs, preferably at least 150 consecutive base pairs, more preferably at least 200 consecutive base pairs of the sequence as described by any of the SEQ ID No. 1 and having essentially the same enzymatic activity as the sequence shown in SEQ ID No. 2.

Functional equivalents are in particular homologs of said sequence. Homologs when used in reference to conjugated linoleic acid isomerases refers orthologs as well as paralogs of the nucleic acid molecule as shown in SEQ ID No.1. These orthologs or paralogs encoding for proteins sharing more than 60%, preferably 65%, 70%, 75%, 80%, more preferably 85%, 90%, 95% or most preferably more than 95% sequence identity on amino acid level with SEQ ID No. 2, wherein said identity is determined over a sequence of at least 100 consecutive amino acids, preferably at least 150 consecutive amino acids, more preferably at least 200 consecutive amino acids of the sequence as described by any of the SEQ ID No. 2 and having essentially the same enzymatic activity as the sequence shown in SEQ ID No. 2.

Functional equivalents as described above might have, compared to the trans-10, cis-12 conjugated linoleic acid isomerase from Propionibacterium acnes (SEQ ID No.1) a reduced or increased enzymatic activity or bioconversion rate. In this context, the enzymatic activity or bioconversion rate of the functional equivalent is at least 50% higher, preferably at least 100% higher, especially preferably at least 300% higher, very especially preferably at least 500% higher than a reference value obtained with the trans-10, cis-12 conjugated linoleic acid isomerase from Propionibacterium acnes (SEQ ID No.1) under otherwise unchanged conditions.

Functionally linked or operably linked: is to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfill its intended function to allow, modify, facilitate or otherwise influence expression of said nucleic acid sequence. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions that are further away, or indeed from other DNA molecules. The terms functionally linked, “operably linked,” “in operable combination,” and “in operable order” as used herein with reference to a conjugated linoleic acid isomerase refers to the linkage of at least one of isomerase to a nucleic acid sequences in a way that the isomerase can be produced or synthesized in the host cell harbouring said DNA molecule. Expression constructs, wherein the trans-10, cis-12 conjugated linoleic acid isomerase from Propionibacterium acnes (SEQ ID No.1) is functionally linked to an promoter are shown in the examples. Operable linkage, and an expression cassette, can be generated by means of customary recombination and cloning techniques as are described, for example, in Maniatis T, Fritsch E F and Sambrook J (1989) Molecular Cloning: A Laboratory Manual, Cold Spring Harbor Laboratory, Cold Spring Harbor (NY), in Silhavy T J, Berman M L and Enquist L W (1984) Experiments with Gene Fusions, Cold Spring Harbor Laboratory, Cold Spring Harbor (NY), in Ausubel F M et al. (1987) Current Protocols in Molecular Biology, Greene Publishing Assoc. and Wiley Interscience and in Gelvin et al. (1990) In: Plant Molecular Biology Manual. However, further sequences which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a promoter and a nucleic acid sequence to be expressed, can exist in a vector-integrated form and be inserted into a bacterial genome, for example by transformation.

Gene: refers to a coding region operably linked to appropriate regulatory sequences capable of regulating the expression of the polypeptide in some manner. A gene includes untranslated regulatory regions of DNA (e.g., promoters, enhancers, repressors, etc.) preceding (upstream) and following (downstream) the coding region (open reading frame, ORF) as well as, where applicable, intervening sequences (i.e., introns) between individual coding regions (i.e., exons). Genes may also include sequences located on both the 5′- and 3′-end of the sequences, which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers, which control or influence the transcription of the gene. The 3′-flanking region may contain sequences, which direct the termination of transcription, posttranscriptional cleavage and polyadenylation.

Genome and genomic DNA of an organism: as used herein is the whole hereditary information of an organism that is encoded in the DNA (or, for some viruses, RNA). This includes both the genes and the non-coding sequences. The term “chromosomal DNA” or “chromosomal DNA sequence” is to be understood as the genomic DNA of the cell independent from the cell cycle status. Chromosomal DNA might therefore be organized in different forms, they might be condensed or uncoiled. An insertion into the chromosomal DNA can be demonstrated and analyzed by various methods known in the art like e.g., polymerase chain reaction (PCR) analysis, Southern blot analysis, fluorescence in situ hybridization (FISH), and in situ PCR.

Heterologous: with respect to a nucleic acid sequence refers to a nucleotide sequence, which is ligated to a nucleic acid sequence to which it is not ligated in nature, or to which it is ligated at a different location in nature.

Hybridizing: as used herein includes “any process by which a strand of nucleic acid joins with a complementary strand through base pairing.” (Coombs 1994, Dictionary of Biotechnology, Stockton Press, New York N.Y.). Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids. As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41 (% G+C), when a nucleic acid is in aqueous solution at 1 M NaCl [see e.g., Anderson and Young, Quantitative Filter Hybridization, in Nucleic Acid Hybridization (1985)]. Other references include more sophisticated computations, which take structural as well as sequence characteristics into account for the calculation of Tm. The person skilled in the art knows well that numerous hybridization conditions may be employed to comprise either low or high stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of either low or high hybridization stringency. Those skilled in the art know that higher stringencies are preferred to reduce or eliminate non-specific binding between the nucleotide sequence of an inventive intron and other nucleic acid sequences, whereas lower stringencies are preferred to detect a larger number of nucleic acid sequences having different homologies to the inventive nucleotide sequences. Such conditions are described by, e.g., Sambrook (Molecular Cloning; A Laboratory Manual, 2nd Edition, Cold Spring Harbor Laboratory Press, Cold Spring Harbor, N.Y. (1989)) or in Current Protocols in Molecular Biology, John Wiley & Sons, N.Y. (1989) 6.3.1-6.3.6. Preferred hybridization condition are disclose in the detailed description.

Identity: when used in relation to nucleic acids refers to a degree of complementarity. Identity between two nucleic acids is understood as meaning the identity of the nucleic acid sequence over in each case the entire length of the sequence, which is calculated by comparison with the aid of the program algorithm GAP (Wisconsin Package Version 10.0, University of Wisconsin, Genetics Computer Group (GCG), Madison, USA) with the parameters being set as follows:

### Gap Weight: 12 Length Weight: 4

### Average Match: 2,912 Average Mismatch: −2,003

For example, a sequence with at least 95% identity to the sequence SEQ ID No. 1 at the nucleic acid level is understood as meaning the sequence that, upon comparison with the sequence SEQ ID No. 1 by the above program algorithm with the above parameter set, has at least 95% identity. There may be partial identity (i.e., partial identity of less then 100%) or complete identity (i.e., complete identity of 100%).

Inducing: when used in relation to the inventive process refers to the inoculation of cell cultures with (i) linoleic acid, or (ii) a expression inducing agent, in the case that the promoter used to drive the expression of a conjugated linoleic acid isomerase is an inducible promoter,

Introducing: with respect to a cell refers to a recombinant DNA expression construct that will be introduced into the bacterial cell. The term introducing encompasses for example methods such as transfection, transduction or transformation.

Isolating: when used in relation to the produced conjugated linoleic acid according to the inventive process refers to the process of extracting (i) the fermentative oil, or (ii) the fatty acid/lipid fraction, or (iii) the conjugated linoleic acid, or (iv) the trans-10, cis-12 conjugated linoleic acid from the fermentation broth, the bacterial pellets/bacterial cell membranes or the supernatant after centrifugation of the fermentation broth (see examples). The isolation can be done from batch-operations or fed-batch-operations. In batch-operations all ingredients used in the operation are fed to the processing vessel at the beginning of the operation and no addition or withdrawal of material takes place during the fermentation process. In fed-batch operations, material can be added or harvested during the fermentation process.

Microorganism: as used herein refers to yeast species and bacteria as defined by Woese (Woese et al., “Towards a natural system of organisms: proposal for the domains Archaea, Bacteria, and Eucarya.” Proc. Natl. Acad. Sci. USA (1990) 87:4576-4579, preferably microorganism that belong to the family selected from the group consisting of Lactobacillaceae, Streptococcaceae, Propionibacteriaceae, Enterobacteriaceae and Bifidobacteriaceae, more preferably the used microorganism belong to the genus selected from the group consisting of Lactococcus, Lactobacillus, Propionibacterium, Escherichia and Bifidobacterium, particularly preferably said microorganism is selected from group consisting of Lactococcus lactis, Lactobacillus paracasei and Escherichia coli to microorganism, including Lactobacillus species, Bifidobacterium species, Lactococcus species and yeasts

Nucleic acid: refers to deoxyribonucleotides, ribonucleotides or polymers or hybrids thereof in single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” can be used to describe a “gene”, “cDNA”, “DNA” “mRNA”, “oligonucleotide” and “polynucleotide”.

Nucleic acid sequence: as used herein refers to the consecutive sequence of deoxyribonucleotides or ribonucleotides (nucleotides) of a DNA fragment (oligonucleotide, polynucleotide, genomic DNA, cDNA etc.) as it can made be available by DNA sequencing techniques as a list of abbreviations, letters, characters or words, which represent nucleotides.

Nucleic acid molecule: as used herein refers to the physically DNA molecule present in the genomic DNA, an appropriate vector or plasmid. The nucleic acid molecule is defined by a nucleic acid sequence.

The term “nutraceutical”: is a combination of “nutritional” and “pharmaceutical” and refers to foods thought to have a beneficial effect on human health. A nutraceutical is any substance that is a food or a part of a food and provides medical or health benefits, including the prevention and treatment of disease. Such products may range from isolated nutrients, dietary supplements and specific diets to genetically engineered designer foods, herbal products, and processed foods such as cereals, soups and beverages

Optical density (or absorbance): of a bacterial culture is the turbidity (optical density) of said culture. For optical density measurements the amount of light with a wavelength of 600 nm that passes through a suspension of cells is determined using a spectrophotometer. The turbidity being, more or less, directly related to cell numbers or mass. The optical density is directly proportional to the cell concentration. Higher optical density is caused by higher bacteria concentrations. In a spectrophotometer, light passing through a sample is measured by a photoelectric cell. As cell density of the sample increases, i.e., becomes more turbid, a greater amount of light is scattered and fails to reach the photoelectric cell. This is measured in terms of optical density (OD) or absorbance (A) units.

OD(A)=log lo/l

where lo=incident light falling on the sample
l=transmitted light; amount of light passing through sample on to the photoelectric cell.

A standard curve can be generated that relates cell numbers or mass to optical density readings, i.e., determine both the optical density and cell numbers (or mass) for a series of samples containing different amounts of microorganisms. Generally the optical density of a sample is directly related to cell density. (In a Klett-Summerson colorimeter, 1 A unit=500 Klett units).

| I (transmitted light) | OD/A | Klett | Cells/ml |
|---|---|---|---|
| 100% | 0.00 | | |
| 90% | 0.045 | 23 | 1 × 10⁸ |
| 75% | 0.125 | 62 | 3 × 10⁸ |
| 50% | 0.30 | 150 | 8.5 × 10⁸ |
| 25% | 0.60 | 300 | 2.2 × 10⁹ |
| 10% | 1.00 | 500 | >4 × 10⁹ |

Otherwise unchanged conditions: means—for example—that the expression which is initiated by one of the expression constructs to be compared is not modified by combination with additional genetic control sequences, for example enhancer sequences and is done in the same environment (e.g., the same plant species) at the same developmental stage and under the same growing conditions.

Probiotics: are defined as live microorganisms, including Lactobacillus species, Bifidobacterium species, Lactococcus species and yeasts, that may beneficially affect the host upon ingestion by improving the balance of the intestinal microflora.

The following describe the various bacteria and yeasts used as probiotics:

### Bifidobacterium

Bifidobacteria are normal inhabitants of the human and animal colon. Newborns, especially those that are breast-fed, are colonized with bifidobacteria within days after birth. Bifidobacteria were first isolated from the feces of breast-fed infants. The population of these bacteria in the colon appears to be relatively stable until advanced age when it appears to decline. The bifidobacteria population is influenced by a number of factors, including diet, antibiotics and stress. Bifidobacteria are gram-positive anaerobes. They are non-motile, non-spore forming and catalase-negative. They have various shapes, including short, curved rods, club-shaped rods and bifurcated Y-shaped rods. Their name is derived from the observation that they often exist in a Y-shaped or bifid form. The guanine and cytosine content of their DNA is between 54 mol % and 67 mol %. They are saccharolytic organisms that produce acetic and lactic acids without generation of CO₂, except during degradation of gluconate. They are also classified as lactic acid bacteria (LAB). To date, 30 species of bifidobacteria have been isolated. Bifidobacteria used as probiotics include Bifidobacterium adolescentis, Bifidobacterium bifidum, Bifidobacterium animalis, Bifidobacterium thermophilum, Bifidobacterium breve, Bifidobacterium longum, Bifidobacterium infantis and Bifidobacterium lactis. Specific strains of bifidobacteria used as probiotics include Bifidobacterium breve strain Yakult, Bifidobacterium breve RO70, Bifidobacterium lactis Bb12, Bifidobacterium longum RO23, Bifidobacterium bifidum RO71, Bifidobacterium infantis RO33, Bifidobacterium longum BB536 and Bifidobacterium longum SBT-2928.

### Lactobacillus

Lactobacilli are normal inhabitants of the human intestine and vagina. Lactobacilli are gram-positive facultative anaerobes. They are non-spore forming and non-flagellated rod or coccobacilli. The guanine and cytosine content of their DNA is between 32 mol % and 51 mol %. They are either aerotolerant or anaerobic and strictly fermentative. In the homofermentative case, glucose is fermented predominantly to lactic acid. Lactobacilli are also classified as lactic acid bacteria (LAB). To date, 56 species of the genus Lactobacillus have been identified. Lactobacilli used as probiotics include Lactobacillus acidophilus, Lactobacillus brevis, Lactobacillus bulgaricus, Lactobacillus casei, Lactobacillus cellobiosus, Lactobacillus crispatus, Lactobacillus curvatus, Lactobacillus fermentum, Lactobacillus GG (Lactobacillus rhamnosus or Lactobacillus casei subspecies rhamnosus), Lactobacillus gasseri, Lactobacillus johnsonii, Lactobacillus plantarum and Lactobacillus salivarus. Lactobacillus plantarum 299v strain originates from sour dough. Lactobacillus plantarum itself is of human origin. Other probiotic strains of Lactobacillus are Lactobacillus acidophilus BG2FO4, Lactobacillus acidophilus INT-9, Lactobacillus plantarum ST31, Lactobacillus reuteri, Lactobacillus johnsonii LA1, Lactobacillus acidophilus NCFB 1748, Lactobacillus casei Shirota, Lactobacillus acidophilus NCFM, Lactobacillus acidophilus DDS-1, Lactobacillus delbrueckii subspecies delbrueckii, Lactobacillus delbrueckii subspecies bulgaricus type 2038, Lactobacillus acidophilus SBT-2062, Lactobacillus brevis, Lactobacillus salivarius UCC 118 and Lactobacillus paracasei subsp paracasei F19.

### Lactococcus

Lactococci are gram-positive facultative anaerobes. They are also classified as lactic acid bacteria (LAB). Lactococcus lactis (formerly known as Streptococcus lactis) is found in dairy products and is commonly responsible for the souring of milk. Lactococci that are used or are being developed as probiotics include Lactococcus lactis, Lactococcus lactis subspecies cremoris (Streptococcus cremoris), Lactococcus lactis subspecies lactis NCDO 712, Lactococcus lactis subspecies lactis NIAI 527, Lactococcus lactis subspecies lactis NIAI 1061, Lactococcus lactis subspecies lactis biovar diacetylactis NIAI 8 W and Lactococcus lactis subspecies lactis biovar diacetylactis ATCC 13675.

Promoter, promoter element, or promoter sequence: as used herein, refers to a DNA sequence which when ligated to a nucleotide sequence of interest is capable of controlling the transcription of the nucleotide sequence of interest into mRNA. Thus, a promoter is a recognition site on a DNA sequence that provide an expression control element for a gene and to which RNA polymerase specifically binds and initiates RNA synthesis (transcription) of that gene. A promoter is typically, though not necessarily, located 5′ (i.e., upstream) of a nucleotide sequence of interest (e.g., proximal to the transcriptional start site of a structural gene). The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any physiological conditions of a cell. In contrast, a “regulatable” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus. A promoter sequence functioning in bateria is understood as meaning, in principle, any promoter which is capable of governing the expression of genes, in particular foreign genes, in bacteria cells. In this context, expression can be, for example, constitutive, inducible or development-dependent. A constitutive promoter is a promoter where the rate of RNA polymerase binding and initiation is approximately constant and relatively independent of external stimuli. Usable promoters are constitutive promoters, such as cos, tac, trp, tet, trp-tet, lpp, lac, Ipp-lac, lacI^{q,} T7, T5, T3, gal, trc, ara, SP6, λ-P_{R} or in the λ-P_{L} promoter, all of which are advantageously used in Gram-negative bacteria. Other advantageous regulatory sequences are contained, for example, in the Gram-positive promoters amy and SPO2, in the yeast or fungal promoters ADC1, MFα, AC, P-60, CYC1, GAPDH, TEF, rp28, ADH. In principle, all natural bacterial promoters with their regulatory sequences as those mentioned above may be used for the process according to the invention. In addition, synthetic promoters may also advantageously be used.

Polypeptide, peptide, oligopeptide, gene product, expression product and protein: are used interchangeably herein to refer to a polymer or oligomer of consecutive amino acid residues.

Recombinant or transgenic DNA expression construct: with respect to, for example, a nucleic acid sequence (expression construct, expression cassette or vector comprising said nucleic acid sequence) refers to all those constructs originating by experimental manipulations in which either
a) said nucleic acid sequence, or
b) a genetic control sequence linked operably to said nucleic acid sequence (a), for example a promoter, or
c) (a) and (b)
   is not located in its natural genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the nucleic acid sequence is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenesis. Such methods have been described (U.S. Pat. No. 5,565,350; WO 00/15815). Recombinant polypeptides or proteins: refer to polypeptides or proteins produced by recombinant DNA techniques, i.e., produced from cells transformed by an exogenous recombinant DNA construct encoding the desired polypeptide or protein. Recombinant nucleic acids and polypeptide may also comprise molecules which as such does not exist in nature but are modified, changed, mutated or otherwise manipulated by man. In one embodiment of the present invention, the recombinant DNA expression construct confers expression of one or more nucleic acid molecules. Said recombinant DNA expression construct according to the invention advantageously encompasses a promoter functioning in bacteria, additional regulatory or control elements or sequences functioning in bacteria and a terminator functioning in bacteria. Additionally, the recombinant expression construct might contain additional functional elements such as expression cassettes conferring expression of e.g. positive and negative selection markers, reporter genes, recombinases or endonucleases effecting the production, amplification or function of the expression cassettes, vectors or recombinant organisms according to the invention. Furthermore, the recombinant expression construct can comprise nucleic acid sequences homologous to a bacterial gene of interest having a sufficient length in order to induce a homologous recombination (HR) event at the locus of the gene of interest after introduction in the bacteria. A recombinant transgenic expression cassette of the invention (or a transgenic vector comprising said transgenic expression cassette) can be produced by means of customary recombination and cloning techniques as are described (for example, in Maniatis 1989, Molecular Cloning: A Laboratory Manual, 2nd Ed., Cold Spring Harbor Laboratory, Cold Spring Harbor (NY); Silhavy 1984,) Experiments with Gene Fusions, Cold Spring Harbor Laboratory, Cold Spring Harbor, N.Y.; and in Ausubel 1987, Current Protocols in Molecular Biology, Greene Publishing Assoc. and Wiley Interscience). The introduction of an expression cassette according to the invention into a bacteria can be effected advantageously using vectors, which comprise the above described nucleic acids, promoters, terminators, regulatory or control elements and functional elements.

Regulatory sequence: refers to promoters, enhancer or other segments of DNA where regulatory proteins such as transcription factors bind and thereby influencing the transcription rate of a given gene.

The term rumen bacteria: refers to those bacteria that can be isolated from the rumen or gastrointestinal tract of ruminant animals (sheep, goats, cattle, deer, etc) where a large part of their digestive process is performed by bacteria.

Structural gene: as used herein is intended to mean a DNA sequence that is transcribed into mRNA which is then translated into a sequence of amino acids characteristic of a specific polypeptide.

Transforming or transformation: as used herein refers to the introduction of genetic material (e.g., a transgene) into a cell. Transformation of a cell may be stable or transient. The term “transient transformation” or “transiently transformed” refers to the introduction of one or more transgenes into a cell in the absence of integration of the transgene into the host cell's genome. The term “transient transformant” refers to a cell which has transiently incorporated one or more transgenes. In contrast, the term “stable transformation” or “stably transformed” refers to the introduction and integration of one or more transgenes into the genome of a cell, preferably resulting in chromosomal integration and stable heritability. Stable transformation of a cell may be detected by Southern blot hybridization of genomic DNA of the cell with nucleic acid sequences, which are capable of binding to one or more of the transgenes. Alternatively, stable transformation of a cell may also be detected by the polymerase chain reaction of genomic DNA of the cell to amplify transgene sequences. The term “stable transformant” refers to a cell that has stably integrated one or more transgenes into the genomic DNA. Thus, a stable transformant is distinguished from a transient transformant in that, whereas genomic DNA from the stable transformant contains one or more transgenes, genomic DNA from the transient transformant does not contain a transgene. Transformation also includes introduction of genetic material into bacteria cells in the form of vectors involving extrachromosomal replication and gene expression. These vectors can be replicated autonomously in the host organism.

Transgenic or recombinant: when used in reference to a cell refers to a cell which contains a transgene, or whose genome has been altered by the introduction of a transgene. Transgenic cells may be produced by several methods including the introduction (as defined above) of a “transgene” comprising nucleic acid (usually DNA) into a target cell or integration of the transgene into a chromosome of a target cell by way of human intervention, such as by the methods described herein. In case of ruminants, the skilled worker can find suitable methods in the following publications:
Gregg, K., Teather, R. M. (1992) The genetic manipulation of rumen bacteria. in “Manipulation of rumen microorganisms”. Ed. K. El-Shazly. Alphagraph, Alexandria, Egypt. pp 1-12.
Gregg, K., Schafer, D., Cooper, C., Allen, G. (1995) Genetic manipulation of rumen bacteria: now a reality. in “Rumen Ecology Research Planning. Eds J. Wallace, A. Lahlou-Kassi. Intl. Livestock. Res. Inst. Nairobi, Kenya. pp. 227-240.
Beard, C. E., Hefford, M. A., Forster, R. J., Sontakke, S., Teather, R. M., Gregg, K. (1995) Stable and efficient transformation system for Butyrivibrio fibrisolvens OB156. Current Microbiol. 30:105-109.
Gregg, K., Allen, G., Beard, C. (1996) Genetic manipulation of rumen bacteria: from potential to reality. Aust. J. Agric. Res. 47:247-256.
Wong, C. M., Klieve, A. V., Hamdorf, B. J., Schafer, D. J., Brau, L., Seet, S. G. M. Gregg, K. (2003) Family of shuttle vectors for ruminal Bacteroides. J. Mol. Microbiol. Biotech. 5: 57-66.

In case of Lactococcus and Lactobacillus, the skilled worker can find suitable methods in the following publications:
Electrocompetent L. lactis were prepared and transformed according to the method described by de Ruyter et al. (1996), while electrocompetent Lb. paracasei NFBC 338 cells were prepared using 3.5×SMEB (1M sucrose, 3.5 mM MgCl₂) as described by Luchansky et al. (1988). Sequence analysis was performed using DNAStar software (DNAStar, Madison, Wis., USA). de Ruyter, P. G., O. P. Kuipers, and W. M. de Vos. 1996. Controlled gene expression systems for Lactococcus lactis with the food-grade inducer nisin. Appl Environ Microbiol 62:3662-7.
Luchansky, J. B., P. M. Muriana, and T. R. Klaenhammer. 1988. Application of electroporation for transfer of plasmid DNA to Lactobacillus, Lactococcus, Leuconostoc, Listeria, Pediococcus, Bacillus, Staphylococcus, Enterococcus and Propionibacterium. Mol Microbiol 2:637-46.

Treatment: as used herein with respect to cancer treatment refers to the therapeutical application of a medicament comprising fermentative oil, preferably purified conjugated linoleic acid, more preferably trans-10, cis 12 octadecadienoic acid produced using the inventive process. Said therapeutical application is to be understood in a broad sense and comprises e.g. the application of said medicament in order to (i) prevent the formation of cancer cells, (ii) reduce or stop the growth of cancer cells and/or (iii) prevent the spread of cancer cells throughout the body

Wild-type, natural or of natural origin: means with respect to an organism, polypeptide, or nucleic acid sequence, that said organism polypeptide, or nucleic acid sequence is naturally occurring or available in at least one naturally occurring organism polypeptide, or nucleic acid sequence which is not changed, mutated, or otherwise manipulated by man.

Vector: is a DNA molecule capable of replication in a host cell. Plasmids and cosmids are exemplary vectors. Furthermore, the terms “vector” and “vehicle” are used interchangeably in reference to nucleic acid molecules that transfer DNA segment(s) from one cell to another, whereby the cells not necessarily belonging to the same organism (e.g. transfer of a DNA segment form an Agrobacterium cell to a plant cell).

The term “expression vector” as used herein refers to a recombinant DNA molecule containing a desired coding sequence and appropriate nucleic acid sequences necessary for the expression of the operably linked coding sequence in a particular host organism.

### DETAILED DESCRIPTION OF THE INVENTION

The teaching of the present invention enables the production of trans-10, cis 12 octadecadienoic acid in transgenic microorganism.

A first embodiment of the present invention relates to a process for the production of trans-10, cis 12 conjugated linoleic acid in a transgenic microorganism comprising the steps of:
(a) introducing into said microorganism at least one nucleic acid molecule encoding a trans-10, cis 12 conjugated linoleic acid isomerase,
(b) culturing the transgenic microorganism obtained under (a),
(c) inducing the production of trans-10, cis 12 conjugated linoleic acid by adding linoleic acid to the culture,
(d) incubating the induced culture for at least 12 hours, and
(e) isolating the conjugated linoleic acid from the culture media and/or microorganism.

In a preferred embodiment, the invention relates to a process for the production of conjugated linoleic acid in a transgenic microorganism according to the above described steps (a) to (e), characterized in that the produced conjugated linoleic acid is a mixture of different CLA isoforms comprising at least 30%, preferably at least 40%, more preferably at least 50%, especially preferably at least 60%, very especially preferably at least 70%, most preferably at least 80% of trans-10, cis 12 octadecadienoic acid.

In addition, the isomeric purity of the trans-10, cis 12 octadecadienoic acid can advantageously be further increased by methods known to the skilled artisan e.g. crystallization.

In a furthermore preferred embodiment of the invention the nucleic acid molecule introduced into the microorganism as described under (a) encodes for a polypeptide with conjugated linoleic acid isomerase activity which is able to convert linoleic acid (9 cis, 12 cis-octadecadienoic acid) to trans-10, cis 12 octadecadienoic acid and can be selected from the following:
i. a nucleic acid molecule having the sequence as described in SEQ ID No. 1, or
ii. from functional equivalents of the polypeptide encoded by the nucleic acid molecule described in (i) such as:
   a. a nucleic acid molecule having at least 50, preferably at least 75, more preferably at least 100, especially preferably at least 125, very especially preferably at least 150 consecutive base pairs of the sequence described by SEQ ID No.1, or
   b. a nucleic acid molecule having an identity of at least 80%, preferably at least 85%, more preferably at least 90%, especially preferably at least 95%, very especially preferably at least 98% over a sequence of at least 100, preferably at least 125, more preferably at least 150, especially preferably at least 175, very especially preferably at least 200 consecutive nucleic acid base pairs to the sequence described by SEQ ID No. 1, or
   c. a nucleic acid molecule hybridizing under high stringent conditions with a nucleic acid fragment of at least 50, preferably at least 100, more preferably at least 150, especially preferably at least 200, very especially preferably at least 500 consecutive base pairs of a nucleic acid molecule described by SEQ ID No. 1, or
   d. a nucleic acid molecule encoding a polypeptide having at least 75%, preferably at least 85%, more preferably at least 90%, especially preferably at least 95%, very especially preferably at least 98% identity to the amino acid sequence as shown in SEQ ID No. 2.

The nucleic acid sequences as defined in ((i) and (ii)) can in principle be identified and isolated from all microorganisms. SEQ ID No. 1 or its homologs/functional equivalents can advantageously be isolated from bacteria, preferrably those bacteria able to produce conjugated fatty acids. Bacteria which may be mentioned are Gram-negative and Gram-positive bacteria. The nucleic acid molecules according to the invention are preferably isolated by methods known to the skilled worker from Gram-positive bacteria such as Propionibacterium, Lactococcus, Bifidobacterium or Lactobacillus, advantageously from Bifidobacterium.

Functional derivatives of the sequence given in SEQ ID No.1 are furthermore to be understood as meaning, for example, allelic variants having at least 75%, preferably at least 85%, more preferably at least 90%, especially preferably at least 95%, very especially preferably at least 98% identity. The identity was calculated as described in the general definitions or by using additional computer programs like PileUp (J. Mol. Evolution., 25 (1987), 351-360, Higgins et al., CABIOS, 5 1989: 151-153). The amino acid sequence derived from the above-mentioned nucleic acid is described by sequence SEQ ID No. 2. Allelic variants encompass, in particular, functional variants which can be obtained from the sequence shown in SEQ ID No. 1 by means of deletion, insertion or substitution of nucleotides, the enzymatic activity of the derived synthetic proteins being retained.

Functional equivalents of the above-described conjugated linoleic acid isomerase can be identified via homology searches in nucleic acid databases or via DNA hybridization (screening of genomic DNA libraries) using a fragment of at least 50 preferably at least 100, more preferably at least 150, especially preferably at least 200, very especially preferably at least 500 consecutive base pairs of the nucleic acid molecule described by the SEQ ID No. 1 and stringent hybridization conditions. In a preferred embodiment of the present invention the stringent hybridizing conditions can be chosen as follows:

The hybridization puffer contains Formamide, NaCl and PEG 6000 (Polyethyleneglykol MW 6000). Formamide has a destabilizing effect on double strand nucleic acid molecules, thereby, when used in hybridization buffer, allowing the reduction of the hybridization temperature to 42° C. without reducing the hybridization stringency. NaCl has a positive impact on the renaturation-rate of a DNA duplex and the hybridization efficiency of a DNA probe with its complementary DNA target. PEG increases the viscosity of the hybridization buffer, which has in principle a negative impact on the hybridization efficiency. The composition of the hybridization buffer is as follows:

250 mM Sodium phosphate-buffer pH 7.2
1 mM EDTA (ethylenediaminetetraacetic acid)
7% SDS (g/v) (sodium dodecyl sulfate)
250 mM NaCl (Sodiumchloride)
10 μg/ml single stranded DNA
5% Polyethylenglykol (PEG) 6000
40% Formamide

The hybridization is preferably performed over night at 42° C. In the morning, the hybridized filter will be washed 3× for 10 minutes with 2×SSC+0.1% SDS. Hybridization should advantageously be carried out with fragments of at least 50, 60, 70 or 80 bp, preferably at least 90 bp. In an especially preferred embodiment, the hybridization should be carried out with the entire nucleic acid sequence with conditions described above.

The skilled worker can find further information on hybridization in the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

The amino acid sequences according to the invention are to be understood as meaning proteins which contain an amino acid sequence shown in SEQ ID No. 2 or a sequence obtainable therefrom by the substitution, inversion, insertion or deletion of one or more amino acid residues, the enzymatic activity of the protein shown in SEQ ID No. 2 being retained or not reduced substantially. The term not reduced substantially is to be understood as meaning all enzymes which still have at least 10%, preferably 20%, especially preferably 30% of the enzymatic activity of the starting enzyme. For example, certain amino acids may be replaced by others with similar physico-chemical properties (spatial dimension, basicity, hydrophobicity and the like). For example, arginine residues are exchanged for lysine residues, valine residues for isoleucine residues or aspartic acid residues for glutamic acid residues. Alternatively, it is possible to exchange the sequence of, add or remove one or more amino acids, or two or more of these measures may be combined with each other.

In a particularly preferred embodiment said trans-10, cis-12 conjugated linoleic acid isomerase is isolated from a rumen bacteria, preferably from Megashera elsdenii YJ-4. In a furthermore particularly preferred embodiment of the invention the nucleic acid molecule encoding trans-10, cis-12 conjugated linoleic acid isomerase is isolated from a Propionibacterium, preferably from Propionibacterium acnes.

In a very particularly preferred embodiment of the current invention said trans-10, cis-12 conjugated linoleic acid isomerase is the trans-10, cis-12 conjugated linoleic acid isomerase with the accession no. CQ766028 isolated from Propionibacterium acnes (SEQ ID No. 1).

In an preferred embodiment of the invention the above described nucleic acid molecule is part of an recombinant or transgenic DNA expression construct (as defined in the general definitions). The recombinant or transgenic DNA expression construct is to be understood as meaning the sequence given in SEQ ID No. 1, or functional equivalents of the polypeptide encoded by the nucleic acid molecule described by SEQ ID No. 1 (as defined above in (ii)) which have been linked functionally to one or more regulatory signals, advantageously for increasing gene expression). These regulatory sequences are, for example, sequences to which inductors or repressors bind and thus regulate the expression of the nucleic acid. In addition to these novel regulatory sequences, or instead of these sequences, the natural regulation of these sequences upstream of the actual structural genes may still be present and, if desired, may have been genetically altered in such a way that the natural regulation has been switched off and the expression of the genes increased. However, the expression of the gene construct may also have a simpler structure, viz. no additional regulatory signals have been inserted upstream of the sequence or its derivatives and the natural promoter with its regulation has not been removed. Instead, the natural regulatory sequence has been mutated in such a way that regulation no longer takes place and gene expression is increased. These altered promoters may also be placed upstream of the natural gene on their own, in order to increase activity. In addition, the gene construct can also advantageously contain one or more so-called enhancer sequences functionally linked to the promoter, and these allow an increased expression of the nucleic acid sequence. It is also possible to insert, at the 3′ end of the DNA sequences, additional advantageous sequences such as further regulatory elements or terminators. One or more copies of the conjugated linoleic acid isomerase gene may be contained in the gene construct.

Advantageous regulatory sequences for the process according to the invention are contained, for example, in promoters such as cos, tac, trp, tet, trp-tet, Ipp, lac, Ipp-lac, lacI^{q,} T7, T5, T3, gal, trc, ara, SP6, λ-P_{R} or in the λ-P_{L} promoter, all of which are advantageously used in Gram-negative bacteria. Other advantageous regulatory sequences are contained, for example, in the Gram-positive promoters amy and SPO2, in the yeast or fungal promoters ADC1, MFα, AC, P-60, CYC1, GAPDH, TEF, rp28, ADH.

In principle, all natural promoters with their regulatory sequences as those mentioned above may be used for the process according to the invention. In addition, synthetic promoters may also advantageously be used.

said recombinant or transgenic DNA expression construct advantageously contains, for expression of the genes present, in addition 3′ and/or 5′ terminal regulatory sequences to increase expression, these being selected for optimal expression depending on the selected host organism and gene or genes.

These regulatory sequences are intended to make specific gene expression possible. This may mean, for example depending on the host organism, that the gene is expressed or overexpressed only after induction, or that it is expressed and/or overexpressed immediately.

The regulatory sequences or factors may for this purpose preferably have a beneficial effect on expression of the introduced genes, and thus increase it. Thus, an enhancement of the regulatory elements can advantageously take place at the level of transcription, by using strong transcription signals such as promoters and/or enhancers. However, it is also possible to enhance translation by, for example, improving the stability of the RNA.

The recombinant or transgenic DNA expression construct may also contain further genes to be introduced into organisms. These genes can be under separate regulation or under the same regulatory region as the isomerase gene according to the invention. These genes are, for example, other biosynthesis genes, advantageously of the fatty acid and lipid biosynthesis, which allow increased synthesis of the isomerase starting material such as linoleic acid.

For optimal expression of heterologous genes in organisms it is advantageous to modify the nucleic acid sequences in accordance with the specific codon usage of the organism. The codon usage can easily be established on the basis of computer analyses of other, known genes of the relevant organism.

For expression in a host organism, for example a microorganism such as yeasts or bacteria, the nucleic acid fragment is advantageously inserted into a vector such as, for example, a plasmid, a phage or other DNA, which vector allows optimal expression of the genes in the host. Examples of suitable plasmids are, in E. coli, pLG338, pACYC184, pBR322, pUC18, pUC19, pKC30, pRep4, pHS1, pHS2, pPLc236, pMBL24, pLG200, pUR290, pIN-III¹¹³-B1, λgt11 or pBdCl, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, in yeasts 2∝M, pAG-1, YEp6, YEp13 or pEMBLYe23, or derivatives of the above-mentioned plasmids. The plasmids mentioned represent a small selection of the plasmids which are possible. Other plasmids are well known to the skilled worker and can be found, for example, in the book Cloning Vectors (Eds. Pouwels P. H. et al. Elsevier, Amsterdam-New York-Oxford, 1985, ISBN 0 444 904018). Suitable plant vectors are described, inter alia, in “Methods in Plant Molecular Biology and Biotechnology” (CRC Press), Chapter 6/7, pp. 71-119.

In addition to plasmids, vectors are also to be understood as meaning all the other vectors which are known to the skilled worker, such as, for example, phages, IS elements, linear or circular DNA. These vectors can be replicated autonomously in the host organism or replicated chromosomally. Autonomous replication is preferred.

The vector advantageously contains at least one copy of the nucleic acid sequence according to the invention. To express the other genes contained, the nucleic acid fragment advantageously additionally contains 3′- and/or 5′-terminal regulatory sequences to increase expression, these sequences being selected for optimal expression, depending on the host organism chosen and the gene or genes.

These regulatory sequences should allow the targeted expression of the gene. Depending on the host organism, this may mean, for example, that the gene is expressed and/or overexpressed only after induction, or that it is expressed and/or overexpressed immediately.

The regulatory sequences or factors can preferably have a positive effect on, and thus increase, the gene expression of the genes introduced. Thus, strengthening of the regulatory elements can advantageously take place at the transcriptional level by using strong transcription signals such as promoters and/or enhancers. In addition, however, strengthening of translation is also possible, for example by improving mRNA stability.

In a further embodiment the gene construct according to the invention can advantageously also be introduced into the organisms in the form of a linear DNA and integrated into the genome of the host organism by means of heterologous or homologous recombination. This linear DNA may consist of a linearized plasmid or only of the nucleic acid fragment as vector or of the nucleic acid sequence according to the invention.

The nucleic acid sequence according to the invention is advantageously cloned into a nucleic acid construct together with at least one reporter gene, and the nucleic acid construct is introduced into the genome. This reporter gene should allow easy detectability via a growth assay, a fluorescence assay, a chemo assay, a bioluminescence assay or a resistance assay, or via a photometric measurement. Examples of reporter genes which may be mentioned are genes for resistance to antibiotics (e.g. ampicillin, chloramphenicol, Tetracyclin, erythromycin) or hydrolase genes, fluorescence protein genes, bioluminescence genes, sugar metabolism genes or nucleotide metabolism genes, or biosynthesis genes such as the Ura3 gene, the IIv2 gene, the luciferase gene, the β-galactosidase gene, the gfp gene, the 2-deoxyglucose-6-phosphate phosphatase gene, the β-glucuronidase gene, the β-lactamase gene, the neomycin phospho-transferase gene or the hygromycin phosphotransferase gene

In a further advantageous embodiment, the nucleic acid sequence according to the invention may also be introduced into an organism on its own.

If it is intended to introduce, into the organism, other genes in addition to the nucleic acid sequence according to the invention, all can be introduced into the organism in a single vector with a reporter gene, or each individual gene with a reporter gene per vector, it being possible for the various vectors to be introduced simultaneously or in succession.

The host organism (=transgenic organism) advantageously contains at least one copy of the nucleic acid according to the invention and/or of the nucleic acid construct according to the invention.

In principle, the nucleic acid according to the invention, the nucleic acid construct or the vector can be introduced into organisms, for example bacteria, by methods known to the skilled worker.

In the case of microorganisms, the skilled worker can find suitable methods in the textbooks by Sambrook, J. et al. (1989) Molecular cloning: A laboratory manual, Cold Spring Harbor Laboratory Press, by F. M. Ausubel et al. (1994) Current protocols in molecular biology, John Wiley and Sons, by D. M. Glover et al., DNA Cloning Vol. 1, (1995), IRL Press (ISBN 019-963476-9), by Kaiser et al. (1994) Methods in Yeast Genetics, Cold Spring Harbor Laboratory Press or by Guthrie et al. Guide to Yeast Genetics and Molecular Biology, Methods in Enzymology, 1994, Academic Press.

Suitable organisms or host organisms (transgenic organisms) for the process according to the invention are, in principle, all organisms which are capable of synthesizing unsaturated fatty acids, and which are suitable for the expression of recombinant genes. Examples which may be mentioned belong to the family selected from the group consisting of Lactobacillaceae, Streptococcaceae, Propionibacteriaceae, Enterobacteriaceae and Bifidobacteriaceae, preferably to the genus selected from the group consisting of Lactococcus, Lactobacillus, Propionibacterium, Escherichia and Bifidobacterium, most preferably said microorganism is selected from group consisting of Lactococcus lactis, Lactobacillus paracasei and Escherichia coli.

The skilled worker knows other suitable sources for the production of fine chemicals, which present also useful nucleic acid molecule sources. They include in general all prokaryotic or eukaryotic cells, preferably unicellular microorganisms, such as fungi like the genus Claviceps or Aspergillus or gram-positive bacteria such as the genera Bacillus, Corynebacterium, Micrococcus, Brevibacterium, Rhodococcus, Nocardia, Caseobacter or Arthrobacter or gram-negative bacteria such as the genera Escherichia, Flavobacterium or Salmonella, or yeasts such as the genera Rhodotorula, Hansenula or Candida.

Production strains which are especially advantageously selected in the process according to the invention are microorganisms selected from the group of the families Actinomycetaceae, Bacillaceae, Brevibacteriaceae, Corynebacteriaceae, Enterobacteriacae, Gordoniaceae, Micrococcaceae, Mycobacteriaceae, Nocardiaceae, Pseudomonaceae, Rhizobiaceae, Streptomycetaceae, Chaetomiaceae, Choanephoraceae, Cryptococcaceae, Cunninghamellaceae, Demetiaceae, Moniliaceae, Mortierellaceae, Mucoraceae, Pythiaceae, Sacharomycetaceae, Saprolegniaceae, Schizosacharomycetaceae, Sodariaceae, Sporobolomycetaceae, Tuberculariaceae, Adelotheciaceae, Dinophyceae, Ditrichaceae and Prasinophyceaeor of the genera and species consisting of Hansenula anomala, Candida utilis, Claviceps purpurea, Bacillus circulans, Bacillus subtilis, Bacillus sp., Brevibacterium albidum, Brevibacterium album, Brevibacterium cerinum, Brevibacterium flavum, Brevibacterium glutamigenes, Brevibacterium iodinum, Brevi-bacterium ketoglutamicum, Brevibacterium lactofermentum, Brevibacterium linens, Brevibacterium roseum, Brevibacterium saccharolyticum, Brevibacterium sp., Coryne-bacterium acetoacidophilum, Corynebacterium acetoglutamicum, Corynebacterium ammoniagenes, Corynebacterium glutamicum (=Micrococcus glutamicum), Coryne-bacterium melassecola, Corynebacterium sp. or Escherichia coli, specifically Escherichia coli K12 and its described strains.

Especially preferred are those bacteria classified or used as probiotics (as defined in the general definitions)

Depending on the host organism, the organisms used in the processes are grown or cultured in the manner known to those skilled in the art. As a rule, microorganisms are grown in a liquid medium which contains a carbon source, usually in the form of sugars, a nitrogen source, usually in the form of organic nitrogen sources such as yeast extract or salts such as ammonium sulfate, a phosphate source such as potassium hydrogen phosphate, trace elements such as iron salts, manganese salts, magnesium salts and, if required, vitamins, at temperatures between 0° C. and 100° C., preferably between 10° C. and 60° C., more preferably between 15° C. and 50° C., while gassing in oxygen. The pH of the liquid medium can be maintained at a fixed value, i.e. the pH is regulated while culture takes place. The pH should then be in a range between pH 2 and pH 9. However, the microorganisms may also be cultured without pH regulation. Culturing can be effected by the batch method, the semi-batch method or fed-batch/continuously. Nutrients may be supplied at the beginning of the fermentation or fed in semicontinuously or continuously.

The organism can be grown under aerobic or anaerobic conditions. The pH of the liquid medium can be maintained at a fixed value, i.e. the pH is regulated while culture takes place. The pH should then be in a range between pH 2 and pH 9, preferably between 4 and 8.5, 4.5 and 8, more preferably between 5 and 7.5, 5.5 and 7. However, the microorganisms may also be cultured without pH regulation

The process according to the invention is advantageously carried out at temperatures between 0° C. and 100° C., preferably between 10° C. and 65° C., 15° C. and 55° C., more preferably between 20° C. and 50° C., 25° C. and 45° C., particularly preferred between 30° C. and 40° C. while gassing in oxygen.

The pH in the process (in vitro) according to the invention is advantageously kept between pH 4 and 12, preferably between 4 and 8.5, 4.5 and 8, more preferably between 5 and 7.5, 5.5 and 7. However, the microorganisms may also be cultured without pH regulation.

A summary of known cultivation methods is to be found in the textbook by Chmiel (Bioprozeβtechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)). The culture medium to be used must meet the requirements of the respective strains in a suitable manner. Descriptions of culture media for various microorganisms are present in the handbook “Manual of Methods for General Bacteriology” of the American Society for Bacteriology (Washington D.C., USA, 1981). These media, which can be employed according to the invention include, as described above, usually one or more carbon sources, nitrogen sources, inorganic salts, vitamins and/or trace elements. Preferred carbon sources are sugars such as mono-, di- or polysaccharides. Examples of very good carbon sources are glucose, fructose, mannose, galactose, ribose, sorbose, ribulose, lactose, maltose, sucrose, raffinose, starch or cellulose. Sugars can also be added to the media via complex compounds such as molasses, or other byproducts of sugar refining. It may also be advantageous to add mixtures of various carbon sources. Other possible carbon sources are oils and fats such as, for example, soybean oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol and/or organic acids such as, for example, acetic acid and/or lactic acid. Nitrogen sources are usually organic or inorganic nitrogen compounds or materials, which contain these compounds. Examples of nitrogen sources include ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as corn steep liquor, soybean meal, soybean protein, yeast extract, meat extract and others. The nitrogen sources may be used singly or as a mixture. Inorganic salt compounds, which may be present in the media include the chloride, phosphorus or sulfate salts of calcium, magnesium, sodium, cobalt, molybdenum, potassium, manganese, zinc, copper and iron.

It is possible to use as phosphorus source phosphoric acid, potassium dihydrogenphosphate or dipotassium hydrogenphosphate or the corresponding sodium-containing salts. Chelating agents can be added to the medium in order to keep the metal ions in solution. Particularly suitable chelating agents include dihydroxyphenols such as catechol or protocatechuate, or organic acids such as citric acid. The fermentation media employed according to the invention for cultivating microorganisms normally also contain other growth factors such as vitamins or growth promoters, which include, for example, biotin, riboflavin, thiamine, folic acid, nicotinic acid, pantothenate and pyridoxine. Growth factors and salts are often derived from complex media components such as yeast extract, molasses, corn steep liquor and the like. Suitable precursors can moreover be added to the culture medium. The exact composition of the media compounds depends greatly on the particular experiment and is chosen individually for each specific case. Information about media optimization is obtainable from the textbook “Applied Microbiol. Physiology, A Practical Approach” (editors P. M. Rhodes, P. F. Stanbury, IRL Press (1997) pp. 53-73, ISBN 0 19 963577 3). Growth media can also be purchased from commercial suppliers such as Standard 1 (Merck) or BHI (Brain heart infusion, DIFCO) and the like. All media components are sterilized either by heat (1.5 bar and 121° C. for 20 min) or by sterilizing filtration. The components can be sterilized either together or, if necessary, separately. All media components can be present at the start of the cultivation or optionally be added continuously or batchwise. The temperature of the culture is normally between 15° C. and 45° C., preferably at 25° C. to 40° C., and can be kept constant or changed during the experiment. The pH of the medium should be in the range from 5 to 8.5, preferably around 7. The pH for the cultivation can be controlled during the cultivation by adding basic compounds such as sodium hydroxide, potassium hydroxide, ammonia or aqueous ammonia or acidic compounds such as phosphoric acid or sulfuric acid. Foaming can be controlled by employing antifoams such as, for example, fatty acid polyglycol esters. The stability of plasmids can be maintained by adding to the medium suitable substances having a selective effect, for example antibiotics. Aerobic conditions are maintained by introducing oxygen or oxygen-containing gas mixtures such as, for example, ambient air into the culture. The temperature of the culture is normally from 20° C. to 45° C. and preferably from 25° C. to 40° C. The culture is continued until formation of the desired product is at a maximum. This aim is normally achieved within 10 hours to 160 hours.

It is possible to use for the process according to the invention growing cells which comprise the nucleic acids, nucleic acid constructs or vectors according to the invention. It is also possible to use resting or disrupted cells. Disrupted cells mean, for example, cells which have been made permeable by treatment with, for example, solvents, or cells which have been ruptured by an enzyme treatment, by a mechanical treatment (for example French press or ultrasound) or by another method. The crude extracts obtained in this way are advantageously suitable for the process according to the invention. Purified or partially purified enzymes can also be used for the process. Likewise suitable are immobilized microorganisms or enzymes which can advantageously be used in the reaction.

If free organisms or enzymes are used for the process according to the invention, these are expediently removed, for example by filtration or centrifugation, before the extraction. It is advantageous that this is unnecessary on use of immobilized organisms or enzymes, but it may still take place.

Linoleic acid as a major starting material can be added to the reaction mixture batchwise, semibatchwise or continuously. The concentration of the starting material for the fermentation process which is preferably linoleic acid is not higher than 3 mg/ml, preferably not higher than 2 mg/ml, more preferably not higher than 1 mg/ml, especially preferably not higher than 0.5 mg/ml. In a very especially preferred embodiment of the current invention the concentration of linoleic acid used to induce the production of trans-10, cis 12 octadecadienoic acid is ranging from 0.1 to 0.5 mg/ml, preferrably from 0.4 to 0.5 mg/ml, more preferrably from 0.3 to 0.4 mg/ml, especially preferrably from 0.2 to 0.3 mg/ml, most preferably from 0.1 to 0.2 mg/ml.

In another preferred embodiment of the invention the linoleic acid is added to a microorganism culture having an optical density (OD₆₀₀) of at least 0.1, preferably of at least 0.2, more preferably of at least 0.3, or 0.31, 0.32, 0.33, 0.34, 0.35, 0.36, 0.37, 0.38, 0.39, especially preferably of at least 0.4, or 0.41, 0.42, 0.43, 0.44, 0.45, 0.46, 0.47, 0.48, 0.49, very especially preferably of at least 0.5, or 0.51, 0.52, 0.53, 0.54, 0.55, 0.56, 0.57, 0.58, 0.59, 0.6. However, the linoleic acid can even be added to microorganism cultures having an optical density (OD₆₀₀) above 0.6.

In a preferred embodiment of the invention, prior to isolation of the CLA, the induced culture is incubated for at least 12 to 18 hours, preferrably for at least 18 to 24 hours, more preferably for at least 24 to 30 hours, especially preferably for at least 30 to 42 hours, most preferably for at least 42 to 72 hours.

With the types of work up mentioned, the product of the process (=conjugated unsaturated fatty acids, especially CLA, preferably trans-10, cis 12 octadecadienoic acid) according to the invention can be isolated in yields of from 20 to 100%, preferably from 30 to 100%, particularly preferably from 50 to 100%, more particularly preferably from 60 to 100%, 70 to 100%, 80 to 100%, 90 to 100%, based on the amount of linoleic acid employed for the reaction. In addition, the products have a high isomeric purity, which can advantageously be further increased where necessary by the crystallization. The inventive process leads to trans-10, cis 12 octadecadienoic acid as major product.

The fatty acids produced can be isolated from the organism by methods with which the skilled worker is familiar. For example via extraction, salt precipitation and/or different chromatography methods. In the case of the fermentation of microorganisms, the abovementioned fatty acids may accumulate in the medium and/or the cells. If microorganisms are used in the process according to the invention, the fermentation broth can be processed after the cultivation. Depending on the requirement, all or some of the biomass can be removed from the fermentation broth by separation methods such as, for example, centrifugation, filtration, decanting or a combination of these methods, or else the biomass can be left in the fermentation broth. The fermentation broth can subsequently be reduced, or concentrated, with the aid of known methods such as, for example, rotary evaporator, thin-layer evaporator, falling film evaporator, by reverse osmosis or by nanofiltration. Afterwards advantageously further compounds for formulation can be added such as corn starch or silicates. This concentrated fermentation broth advantageously together with compounds for the formulation can subsequently be processed by lyophilization, spray drying, spray granulation or by other methods. Preferably the fatty acids or the fatty acid compositions are isolated from the organisms, such as the microorganisms or the culture medium in or on which the organisms have been grown, or from the organism and the culture medium, in the known manner, for example via extraction, distillation, crystallization, chromatography or a combination of these methods. These purification methods can be used alone or in combination with the aforementioned methods such as the separation and/or concentration methods.

The product-containing composition can be subjected for example to a thin layer chromatography on silica gel plates or to a chromatography such as a Florisil column (Bouhours J. F., J. Chromatrogr. 1979, 169, 462), in which case the desired product or the impurities are retained wholly or partly on the chromatography resin. These chromatography steps can be repeated if necessary, using the same or different chromatography resins. The skilled worker is familiar with the choice of suitable chromatography resins and their most effective use. An alternative method to purify the fatty acids is for example crystallization in the presence of urea. These methods can be combined with each other.

The identity and purity of the isolated compound(s) can be determined by prior art techniques. These include high performance liquid chromatography (HPLC), spectroscopic methods, mass spectrometry (MS), staining methods, thin-layer chromatography, NIRS, enzyme assay or microbiological assays. These analytical methods are summarized in: Patek et al. (1994) Appl. Environ. Microbiol. 60:133-140; Malakhova et al. (1996) Biotekhnologiya 11 27-32; and Schmidt et al. (1998) Bioprocess Engineer. 19:67-70. Ulmann's Encyclopedia of Industrial Chemistry (1996) Vol. A27, VCH: Weinheim, pp. 89-90, pp. 521-540, pp. 540-547, pp. 559-566, 575-581 and pp. 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Vol. 17.

In a particularly preferred embodiment the invention relates to a process for the production of conjugated linoleic acid in a transgenic microorganism according to the above described steps (a) to (e), characterized in that the bioconversion rate (as defined in the general definitions) of linoleic acid in the operation or fermentation procedure (batch or fed-batch) is higher than 10%, preferably higher than 20%, more preferably higher than 30% for bacteria belonging to the genus Lactobacillus, higher than 10%, preferably higher than 20%, more preferably higher than 30%, especially preferably higher than 40% for bacteria belonging to the genus Escherichia and higher than 10%, preferably higher than 20%, more preferably higher than 30%, especially preferably higher than 40%, very especially preferably higher than 50% for bacteria belonging to the genus Lactococcus.

Furthermore, the invention relates to a process for the production of feed or food products or nutraceuticals enriched in conjugated linoleic acid, wherein the conjugated linoleic acid is produced according to the above described process.

The invention relates furthermore to feed-, food-products and nutraceuticals enriched in conjugated linoleic acid, wherein the conjugated linoleic acid is produced according to the above described process.

The compositions of the present invention find a wide variety of nutritional, therapeutic and pharmacological uses. These uses include: the reduction of body fat in animals: increasing muscle mass in animals, increasing feed efficiency in animals, reducing body weight in humans, attenuating allergic reactions in animals, preventing weight loss due to immune stimulation in animals, increasing the mineral content of bone in animals, preventing skeletal abnormalities in animals, and decreasing the amount of cholesterol in the blood of animals.

The feed- or food-products, preferably preparations used as additives for feed- or food-products, in addition to the conjugated linoleic acid, preferably the fementated oil or the purified conjugated linoleic acid isomer mixture, more preferrably the purified trans-10, cis 12 octadecadienoic acid, produced according to the above described process, can comprise further constituents. The choice of further constituents will be guided here by the chosen field of use of the preparations and is in general known to the skilled artisan. Further constituents within the meaning of the present invention which come into consideration are, for example, the following substances: further organic acids, carotenoids, trace elements, antioxidants, vitamins, enzymes, amino acids, minerals, emulsifiers, stabilizers, preservatives, anticaking agents and/or flavor enhancers.

Examples of representatives of said substance classes which come into consideration can be taken from the respectively valid lists of food additives according to European regulations, for example the currently valid EC Directive 95/2/EC.

Hereinafter, further constituents suitable for producing inventive preparations are listed:

These constituents are added in different amounts to the preparations according to their different properties and as a function of the chosen field of use. The quantitative mixture ratios and also expedient combinations of the substance classes as a function of the chosen field of use are known to those skilled in the art.

Organic acids which are preferably used are formic acid, propionic acid, lactic acid, acetic acid and citric acid, particular preference being given to formic acid, propionic acid or lactic acid.

In the context of the present invention, carotenoids are taken to mean tetraterpenes in which one or two ionone rings are bonded by a carbon chain having 9 double bonds and can be of either plant or animal origin. Carotenoids are also taken to mean the oxygenated xanthophylls. Those which may be mentioned by way of example are: alpha-, beta-, gamma-carotenes, ixin, norbixin, capsanthin, capsorubin, lycopene, beta-apo-8-carotenal, carotinic acid ethyl ester and also the xanthophylls flavoxanthin, lutein, cryptoaxanthin, rubixanthin, violaxanthin, rhodoxanthin and also canthaxanthin.

The inventive preparations can comprise, for example, the following trace elements: chromium, iron, fluorine, iodine, cobalt, copper, manganese, molybdenum, nickel, selenium, vanadium, zinc or tin.

The E numbers listed hereinafter are the designation used in Directive 95/2/EEC for food additives.

Antioxidants which can be used are, for example, ascorbic acid (vitamin C, E 300), sodium L-ascorbate (E 301), calcium L-ascorbate (E 302), ascorbyl palmitate (E 304), butylated hydroxyanisole (E 320), butylated hydroxytoluene (E 321), calcium disodium EDTA (E 385), gallates, for example propyl gallate (E 310), octyl gallate (E 311), dodecyl gallate (lauryl gallate) (E 312), isoascorbic acid (E 315), sodium isoascorbate (E 316), lecithin (E 322), lactic acid (E 270), multiple phosphates, for example diphosphates (E 450), triphosphates (E 451), polyphosphates (E 452), sulfur dioxide (E 220), sodium sulfite (E 221), sodium bisulfite (E 222), sodium disulfite (E 223), potassium sulfite (E 224), calcium sulfite (E 226), calcium hydrogensulfite (E 227), potassium bisulfite (E 228), selenium, tocopherols (vitamin E, E 306), for example alpha-tocopherol (E 307), gamma-tocopherol (E 308), delta-tocopherol (E 309) and all tocotrienols, tin(II) chloride (E 512), citric acid (E 330), sodium citrate (E 331), carotenoids, vitamin A and also potassium citrate (E 332).

Vitamins which come into consideration are not only fat-soluble vitamins, but also water-soluble vitamins. Examples of fat-soluble vitamins are: vitamin A (retinol), vitamin D (calciferols), vitamin E (tocopherols and tocotrienols), vitamin K (phylloquinones and menaquinones), preference being given to vitamins A and E.

Examples of water-soluble vitamins are: vitamin B1 (thiamine), vitamin B2 (riboflavin), vitamin B5 (pantothenic acid), vitamin B6 (pyridoxin), vitamin B12 (cobalamin), vitamin C (ascorbic acid), vitamin H (biotin), folic acid and niacin, preference being given to the vitamins B2 and C. The preparations can also comprise enzymes. Those which may be mentioned by way of example are: amylases, proteases and invertases.

Amino acids coming into consideration in the context of this invention are, for example, glutamic acid, L-carnitine, L-glutamine, L-taurine, L-aspartic acid, L-glycine, L-lysine, DL-phenylalanine, L-tryptophan, tyrosine, L-arginine, L-cysteine, L-leucine, L-methionine, L-alanine, L-serine, L-threonine, L-citrulline, L-valine, L-histidine, L-isoleucine, L-ornithine or L-proline.

Particular preference is given to the essential amino acids, for example L-isoleucine, L-leucine, L-lysine, L-methionine, DL-phenylalanine, L-threonine, L-tryptophan and L-valine, very particular preference being given to the amino acids important in animal nutrition L-lysine, DL-methionine or L-threonine.

Minerals in the context of this invention are, for example, sodium, potassium, magnesium, calcium, phosphorus, iron and zinc.

As emulsifiers, use can be made of the following substances, for example: E 420 sorbitol, E 420ii sorbitol syrup, E 421 mannitol, E 422 glycerol, E 431 polyoxyethylene(40) stearate, E 432 polyoxyethylene sorbitan monolaurate/Polysorbate 20, E 433 polyoxyethylene sorbitan monooleate/Polysorbate 80, E 434 polyoxyethylene sorbitan monopalmitate/Polysorbate 40, E 435 polyoxyethylene sorbitan monostearate/Polysorbate 60, E 436 polyoxyethylene sorbitan tristearate/Polysorbate 65, E 440 pectins, E 440i pectin, E 440ii amidated pectin, E 442 ammonium phosphatides, E 444 sucrose acetate isobutyrate, E 445 glycerol esters of root rosin, E 450 diphosphates, E 450i disodium diphosphate, E 450ii trisodium diphosphate, E 450iii tetrasodium diphosphate, E 450iv dipotassium diphosphate, E 450v tetrapotassium diphosphate, E 450vi dicalcium diphosphate, E 450vii calcium dihydrogendiphosphate, E 451 triphosphates, E 451i pentasodium triphosphate, E 451ii pentapotassium triphosphate, E 452 polyphosphates, E 452i sodium polyphosphate, E 452ii potassium polyphosphate, E 452iii sodium calcium polyphosphate, E 452iv calcium polyphosphate, E 460 cellulose, E 460i microcrystalline cellulose, E 460ii cellulose powder, E 461 methylcellulose, E 463 hydroxypropylcellulose, E 464 hydroxypropylmethylcellulose, E 465 methylethylcellulose, E 466 carboxymethylcellulose, E 469 enzymatically hydrolyzed carboxymethylcellulose, E 470a sodium salts, potassium salts and calcium salts of fatty acids, E 470b magnesium salts of fatty acids, E 471 mono- and diglycerides of fatty acids, E 472a acetic acid esters of mono- and diglycerides of fatty acids, E 472b lactic acid esters of mono- of diglycerides of fatty acids, E 472c citric acid esters of mono- and diglycerides of fatty acids, E 472d tartaric acid esters of mono- and diglycerides of fatty acids, E 472e mono- and diacetyltartaric acid esters of mono- and diglycerides of fatty acids, E 472f mixed acetic and tartaric acid esters of mono- and diglycerides of fatty acids, E 473 sucrose esters of fatty acids, E 474 sucroglycerides, E 475 polyglycerol esters of fatty acids, E 476 polyglycerol polyricinoleate, E 477 propylene glycol esters of fatty acids, E 479 thermally oxidized soybean oil interacted with mono- and diglycerides of fatty acids, E 481 sodium stearoyl-2-lactylate, E 482 calcium stearoyl-2-lactylate, E 483 stearyl tartrate, E 491 sorbitan monostearate, E 492 sorbitan tristearate, E 493 sorbitan monolaurate, E 494 sorbitan monooleate or E 495 sorbitan monopalmitate.

Stabilizers are substances which maintain the consistency or the composition of foods. Those which may be mentioned by way of example are: ascorbic acid (E 300), carbamide (E 927b), iron(II) lactate (E 585), iron gluconate (E 579), glycerol esters (E 445), lecithin (E 322), metatartaric acid (E 353), pectin (E 440), sucrose acetate isobutyrate (E 444) and tin(II) chloride (E 512).

Preservatives are substances which prolong the shelf life of foods, by protecting them from the harmful effects of microorganisms. Those which may be mentioned by way of example are: E 200 sorbic acid, E 201 sodium sorbate, E 202 potassium sorbate, E 203 calcium sorbate, E 210 benzoic acid, E 211 sodium benzoate, E 212 potassium benzoate, E 213 calcium benzoate, E 214 ethyl p-hydroxybenzoate/PHB ester, E 215 sodium ethyl p-hydroxybenzoate/PHB ethyl ester sodium salt, E 216 propyl p-hydroxybenzoate/PHB propyl ester, E 217 sodium propyl p-hydroxybenzoate/PHB-propyl ester sodium salt, E 218 methyl p-hydroxybenzoate/PHB-methyl ester, E 219 sodium methyl p-hydroxybenzoate/PHB-methyl ester sodium salt, E 220 sulfur dioxide, E 221 sodium sulfite, E 222 sodium hydrogensulfite/sodium bisulfite, E 223 sodium metabisulfite/sodium disulfite, E 224 potassium metabisulfite/potassium sulfite, E 226 calcium sulfite, E 227 calcium hydrogensulfite, E 228 potassium hydrogensulfite/potassium bisulfite, E 230 biphenyl/diphenyl, E 231 orthophenyl phenol, E 232 sodium orthophenyl phenol, E 233 thiabendazole, E 234 nisin, E 235 natamycin, E 239 hexamethylenetetramine, E 242 dimethyl dicarbonate, E 249 potassium nitrite, E 250 sodium nitrite, E 251 sodium nitrate and E 252 potassium nitrate.

Anticaking agents in the context of the present invention are naturally occurring or synthesized substances which increase the flowability of a food by preventing the clumping together and sticking together of the particles. Examples which may be mentioned are: E 530 magnesium oxide, E 535 sodium ferrocyanide, E 536 potassium ferrocyanide, E 541 acidic sodium aluminum phosphate, E 551 silicon dioxide, E 552 calcium silicate, E 553ai magnesium silicate, E 553aii magnesium trisilicate (asbestos free), E 553b talc (asbestos free), E 554 sodium aluminum silicate and E 556 calcium aluminum silicate.

Flavor enhancers in the context of this invention are taken to mean naturally occurring or synthesized substances which are able to round off or enhance the flavor of foods. These also include flavorings. Examples which may be mentioned are: E 620 glutamic acid, E 621 monosodium glutamate, E 622 monopotassium glutamate, E 623 calcium diglutamate, E 624 monoammonium glutamate, E 625 magnesium diglutamate, E 626 guanylic acid, E 627 disodium guanylate, E 628 dipotassium guanylate, E 629 calcium guanylate, E 630 inosinic acid, E 631 disodium inosinate, E 632 dipotassium inosinate, E 633 dicalcium inosinate, E 634 calcium 5-ribonucleotide, E 635 disodium 5-ribonucleotide, E 640 glycine and E 650 zinc acetate.

In one embodiment, the inventively used preparation can comprise aids. Aids are taken according to the invention to mean substances which serve to improve the product properties, such as dusting behavior, flow properties, water absorption capacity and storage stability. Aids can be based on sugars, e.g. lactose or maltose dextrin, based on cereal or legume products, e.g. corn cob meal, wheat bran and soybean meal, based on mineral salts, inter alia salts of calcium, magnesium, sodium or potassium, and also D-pantothenic acid or its salts themselves (D-pantothenic acid salt produced chemically or by fermentation).

In a further embodiment, the inventively used preparations can comprise carriers. Suitable carriers are “inert” carrier materials, that is to say materials which do not display adverse interactions with the components used in the inventive preparation. Obviously, the carrier material must be safe for the respective uses as aid, for example in foods and animal feedstuffs. Suitable carrier materials are not only inorganic carriers but also organic carriers. Examples of suitable carrier materials which may be mentioned are: low-molecular-weight inorganic or organic compounds and also relatively high-molecular-weight organic compounds of natural or synthetic origin. Examples of suitable low-molecular-weight inorganic carriers are salts, such as sodium chloride, calcium carbonate, sodium sulfate and magnesium sulfate, kieselguhr or silicic acid, or silicic acid derivatives, for example silicon dioxides, silicates or silica gels. Examples of suitable organic carriers are, in particular, sugars, for example glucose, fructose, sucrose and also dextrins and starch products. Examples of relatively high-molecular-weight organic carriers which may be mentioned are: starch and cellulose preparations, such as in particular corn starch, corn cob meal, ground rice hulls, wheat semolina bran or cereal flours, for example wheat, rye, barley and oat flour or brans and mixtures thereof.

The inventively used preparations can comprise the further constituents, carriers and aids in mixtures.

The weight fraction of the conjugated linoleic acid in the preparations can vary in wide ranges and is generally orientated according to practical considerations which result from the chosen field of application (for example farm animal husbandry, raising domestic animals or human nutrition).

The preparations are produced in the simplest case by mixing the constituents. Likewise, they can be produced by mixing solutions of the individual components, and if appropriate subsequently removing solvents.

The mixtures of various constituents can be present in any weight ratios to one another.

The simplest form of the mixture is bringing together the constituents in a mixer. Such mixers are known to those skilled in the art, for example from the Ruberg company (vertical twin-shaft mixer (type HM (10-50 000 l)), ring-layer mixer-pelletizer (type RMG), continuous agglomerator dryer (type HMTK), vertical single-shaft mixer (type VM (10-50 000 l)), container mixer (type COM (50-4000 l)). Further mixers can also be obtained from Lödige, Drais, Engelsmann. The mixers can be operated batchwise or continuously. In the batchwise mixer, generally all constituents to be mixed are charged in the desired ratio and then mixed for an adequate time in the region of minutes to hours. The mixing time and the mixing stress are specified so that the constituents are present homogeneously distributed in the mixture. In the case of continuous mixing, the constituents are added continuously, if appropriate after premixing. In the continuous mixer, also, the residence time and mixing stress are to be chosen in such a manner that the constituents are present homogeneously distributed in the mixture. The mixing time is frequently shorter in the continuous case and the stress is higher than in the case of batchwise mixing. The mixing is customarily performed at room temperature, but can also, depending on the substances used, be carried out at higher or lower temperatures.

In a preferred embodiment, the preparations are present in solid form. Depending on the application requirement, the preparations can be powders having a mean particle size of from 10 μm to 5000 μm, preferably having a mean particle size of from 20 μm to 1000 μm.

The resultant particle size distribution of the pulverulent products can be studied in an instrument from Malvern Instruments GmbH, Mastersizer S.

Mixtures of constituents are possible as pure blends, that is to say the substances are mixed together in the desired particle sizes and concentration ratios, if appropriate with addition of further additives, substances also being able to be protected, for example, by a coating if necessary. Furthermore, core-sheath structures can be used, that is to say one constituent is situated on the interior as core and a further constituent as sheath on the outside, or vice versa. Of course, in the case of these structures, further coatings can also be used, if this is necessary. It is also conceivable to encapsulate substances together in a shared matrix of carrier materials or protective colloids. Examples of these are known to those skilled in the art and are described, for example, in R. A. Morten: Fat-Soluble Vitamins, Pergamon Press, 1970, pages 131 to 145.

The powders can be produced by crystallization, precipitation, drying, pelleting or agglomeration methods familiar to those skilled in the art, or other methods for forming solids described in current textbooks.

The exact amount of CLA to be incorporated into a dietetic food depends upon the intended use of the food, the form of CLA employed and the route of administration. It also can depend upon the isomer ratios. However, the dietetic food will contain the equivalent of about 0.05 to about 1%, or about 0.1% to about 0.9%, or 0.2% to about 0.8%, or 0.3% to about 0.7%, or 0.4% to about 0.6% of CLA by weight of the dietetic food. In an additional embodiment the food will contain the equivalent of about 1% to about 10%, or 2% to about 8%, or 3% to about 7%, or 4% to about 6% of CLA by weight of the dietetic food. The CLA content can also be expressed as the amount of CLA based on the total calories in the serving. e.g. 0.03 to 3 gram CLA per 100 calorie serving. Alternatively the amount of CLA can also be expressed as a percentage of the lipid or fat in the food, such as 0.3% to 100% of the food lipid.

Additional suitable feedstuff and/or food containing conjugated linoleic acid are described in the U.S. Pat. No. 6,042,869 (examples 2 to 9) and U.S. Pat. No. 5,760,082 (examples 2 to 5). The cited content of the mentioned Patents is herein incorporated by reference.

Other patents describe various formulations of CLA. European patent application EP779033 A1, herein incorporated by reference, discloses an edible fat spread containing 0.05 to 20% (by weight) CLA residues. There, a commercially-available mixture of free fatty acids having a linoleic acid content of 95.3% was subjected to alkali isomerization with NaOH in ethylene glycol. The free fatty acids were incorporated into triglycerides by mixing with 10 parts palm oil and lipase. The mixture was stirred for 48 hours at 45° C. and the lipase and free fatty acids removed. Seventy parts of this compositions and 29 parts water. 0.5 parts whey protein powder, 0.1 parts sals, and a small amount of flavor and citric acid (to obtain a pH of 4.5) were combined and processed to produce a fat spread. Other dietetic foods containing a safe and effective amount of CLA are disclosed in PCT publication WO 97/46118 (Cook et al.), herein incorporated by reference. There, a liquid dietetic food for parenteral administration to humans containing fat particles of about 0.33-0.5 micrometers in diameter is disclosed. The emulsion contains 0.5 mg/gm to 10 mg/gm of CLA or alternatively, 0.3% to 100% CLA based on the food lipid or 0.03 gm to 0.3 gm CLA per 100 calorie serving. This application also discloses a baby formula containing similar amounts of CLA along with 2.66 gm of protein, 5.46 gm of fat, 10.1 gm of carbohydrate, 133 gm of water, and vitamins and minerals in RDA (Recommended Daily Allowance) amounts. Another example of a low-residue liquid enteral dietetic product useful as a high-protein, vitamin and mineral supplement is disclosed. This supplement contains CLA at 0.05% to about 5% by weight of the product. or by 0.3% to about 100% of the lipid present or about 0.03 to 0.3 gm CLA per 100 calories. Additionally, 140 calories of a representative formula can contain 7.5 gm of egg white solids, 0.1 gm CLA, 27.3 gm carbohydrate such as sucrose or hydrolyzed cornstarch, 1.9 gm of water, and vitamins and minerals in RDA amounts.

Additionally, the invention relates to transgenic microorganism expressing a nucleic acid molecule as described above encoding a trans-10, cis-12 conjugated linoleic acid isomerase characterized by a sequence
(i) a nucleic acid molecule having the sequence as described in SEQ ID No. 1, or
(ii) from functional equivalents of the polypeptide encoded by the nucleic acid molecule described in (i) such as:
   e. a nucleic acid molecule having at least 50, preferably at least 75, more preferably at least 100, especially preferably at least 125, very especially preferably at least 150 consecutive base pairs of the sequence described by SEQ ID No.1, or
   f. a nucleic acid molecule having an identity of at least 80%, preferably at least 85%, more preferably at least 90%, especially preferably at least 95%, very especially preferably at least 98% over a sequence of at least 100, preferably at least 125, more preferably at least 150, especially preferably at least 175, very especially preferably at least 200 consecutive nucleic acid base pairs to the sequence described by SEQ ID No. 1, or
   g. a nucleic acid molecule hybridizing under high stringent conditions with a nucleic acid fragment of at least 50, preferably at least 100, more preferably at least 150, especially preferably at least 200, very especially preferably at least 500 consecutive base pairs of a nucleic acid molecule described by SEQ ID No. 1, or
   h. a nucleic acid molecule encoding a polypeptide having at least 75%, preferably at least 85%, more preferably at least 90%, especially preferably at least 95%, very especially preferably at least 98% identity to the amino acid sequence as shown in SEQ ID No. 2.
      wherein said nucleic acid sequence is preferably isolated from a rumen bacteria, more preferably from Megashera elsdenii, most preferably from Megashera elsdenii YJ-4, or from a microorganism belonging to the genus Propionibacterium, preferably Propionibacterium acnes, wherein said nucleic acid molecule is functionally linked to at least one heterologous promoter sequence.

In a furthermore preferred embodiment the present invention relates to the use of the inventive transgenic microorganism, preferably microorganism belonging to the genus selected from the group consisting of Lactococcus, Lactobacillus, Propionibacterium, Escherichia and Bifidobacterium, as described above, more preferably microorganism selected from the group consisting of Bifidobacterium breve, Bifidobacterium dentium and Bifidobacterium pseudocatenulatum as probiotics in food and feed.

Additionally, the invention relates to fermented oil produced in transgenic microorganism according to the above described inventive process. In a preferred embodiment the fermentative oil is isolated from the fermentation broth and consist mainly of trans-10, cis-12 octadecadienoic acid and 9,12-Octadecadienoic acid and is enriched in trans-10, cis-12 octadecadienoic acid to at least 20%, 30%, 40%, preferably at least 50%, 55%, 60% more preferably at least 65%, 70%, 75% especially preferably at least 80%, 85%, 90% very especially preferably at least 91%, 92%, 93%, 94%, 95%. In order to purify the fatty acid fraction, preferably the CLA fraction, said fermented oil can be further processed (see example).

The invention relates furthermore to the use of the fermented oil produced according to the above described inventive method for
1. attenuating allergic reaction in animals mediated by Type 1 or TgE hypersensitivity by administering CLA in concentrations of about 0.1 to 1.0% to preserve number of white blood cells as described in the U.S. Pat. No. 3,585,400 (Cook et al.), herein incorporated by reference. This patent discloses that guinea pigs fed with 0.25% CLA or control diests for two weeks, then immunized with ovalbumin on weeks two and three for hyperimmunization. A superfusion model system was used to determine if feeding CLA had any effect on the allergen induced tracheal contraction. Trachea from guinea pigs feed with CLA were more stable in the superfusion system than trachea of control-fed guinea pigs. When allergen was infused over the guinea pig trachea, less tracheic contraction was observed in the tissue of the CLA-fed animals. The white blood cell count of animals fed CLA was elevated as compared to control animals, the CLA-fed animals having a white blood cell count Of 3.5×10⁶+/−0.6 as compared to 2.4×10⁶+/−0.3 for the control animals.
2. reducing body fat of animals. U.S. Pat. No. 5,554,646 (Cook et al.), incorporated herein by reference, discloses the use of CLA for reducing body fat in animals. In this method, a safe and effective amount of CLA sufficient to cause reduction of body weight is fed to the animal. Mice fed a diet containing 0.5% CLA had a total fat content at the end of feeding that was significantly lower that the fat content of control mice fed a diet containing 0.5% corn oil. The exact amount of CLA administered to reduce body fat depends upon the animal, the form of CLA employed, and the route of administration. The amount generally ranges from about 0.001 g/kg to about 1 g/kg of the animal body weight.
3. enhancing weight gain and feed efficiency in the animals. Such a nutritive use of CLA is disclosed in U.S. Pat. No. 5,428,072 (Cook et al.). There, feeding a safe and effective amount of CLA to animals is shown to enhance weight gain and feed efficiency in the animal. Groups of chicks fed a diet supplemented with 0.5% CLA demonstrated equivalent weights gain to control chicks fed 0.5% linoleic acid even though the CLA-fed chicks consumed less food.
4. preventing anorexia and weight loss due to immune stimulation. The use of CLA to enhance growth and prevent anorexia and weight loss due to immune stimulation (e.g. endotoxin exposure) and the adverse effects of catabolic hormones (e.g., IL-1) was disclose in U.S. Pat. No. 5,430,066 (Cook et al.) herein incorporated by reference. Chicks fed a diet of 0.5% CLA and subsequently challenged by endotoxin injection exhibited weight gain while chicks fed a control diet failed to gain weight following endotoxin exposure. Similar results were obtained in rats fed a diet containing 0.5% CLA as compared to animals fed a control diet containing 0.5% corn oil. Preparations and dosage ranges disclosed were identical to those disclosed in U.S. Pat. No. 5,554,646.
5. maintaining or elevate CD-4 and CD-8 cell levels in animals. Methods for treating animals to maintain or elevate CD-4 and CD-8 cell levels and to prevent or alleviate the adverse effects on the animal caused by the production or exogenous administration of tumor necrosis factor (TNF) or by a virus consisting of administering to the animal a sage and effective amount of CLA were disclosed in the U.S. Pat. No. 5,674,902 (Cook et al.), herein incorporated by reference. Mice were fed either a control diet or 0.5% CLA and subsequently challenged with injections of TNF. Mice fed CLA lost less weight than the control mice. Likewise, chicks fed a 0.5% CLA diet and subsequently challenged with a wing web injection of live attenuated fowl pox virus gained more weight than chicks fed a control diet. Chicks fed the 0.5% CLA diet demonstrated a markedly enhanced percent of CD-4 and CD-8 cells as compared to chicks fed a control diet.
6. improving blood lipid profile in animals. European Patent Application 779,033 A1 (Lievense et al.), herein incorporated by reference, discloses the use of CLA for improving blood lipid profile. Briefly, hamsters were fed diets containing CLA incorporated onto a triglyceride in the form of a fat spread at a rate of 1.5% of the total calories of their diet. Hamsters fed CLA exhibited a decrease in total cholesterol, a decrease in HDL cholesterol, and decrease in LDL cholesterol.
7. the production of a medicament or therapeutic agents for the treatment of cancer. The anti-proliferative effect of the fermented oils produced by L. lactis and E. coli on human SW480 cancer cells was examined by the inventor of the present invention and the results clearly demonstrate the cytotoxic effect the t10, c12 CLA isomer exert on the cancer cells. Cell growth inhibition by t10, c12 CLA was dose-dependent with highest cytotoxic effect at concentrations of 20 μg/ml t10, c12 CLA. L. lactis t10, c12 CLA killed most of the cancer cells, less than 8% viable cells remained when treated with the highest concentration (20 μg/ml), compared with ethanol control (=100%) and incubation with the fermented t10, c12 CLA produced by E. coli caused a reduction to ˜20%. CLA isomers have previously been reported to decrease cell viability and stimulate apoptosis in SW480 cells. In a study by Miller et al. (2002), the 10, c12 CLA isomer was the most potent isomer, which reduced cell viability by 47-61% compared with 40-52% reduction by the c9, t11 CLA. CLA has also been shown to inhibit growth of the MCF-7 breast cancer cell line (Schultz et al., 1992; O'Shea et al., 1999). When MCF-7 cells were treated with 20 μg/ml of the t10, c12 CLA isomer for 8 days, a 15% decrease in cell numbers was observed, whereas the same amount of the c9, t11 CLA isomer caused a 60% decrease in cell viability during same conditions (O'Shea et al., 1999). In another study, the t10, c12 CLA isomer reduced viability by 50-60% in both SW480 and MCF-7 cell lines following 4 days incubation with 16 μg/ml (Miller et al., 2001). In contrast, the same amount of linoleic acid increased viability of SW480 cells by 23%. Unfermented control linoleic acid and the pure linoleic acid (Sigma) had only a minor effect on the cell viability.

Medicaments and therapeutic agents are taken to mean those agents which are used not only for prevention, but also for therapeutic treatment of allergic reaction, increased body fat, anorexia and weight loss due to immune stimulation, blood lipid profiles and cancer in animals, preferably in human.

In the therapeutic treatment of e.g. allergic reaction, increased body fat, anorexia and weight loss due to immune stimulation, blood lipid profiles and cancer, the preparations can be formulated in a manner which is generally known to those skilled in the art and is suitable and can be used for the production of pharmaceutical dosage forms with the use of conventional techniques. Such techniques are described, for example, in “Remington's Pharmaceutical Science Handbook”, Mack Publishing Co., New York, USA, 17^{th} edition 1985. Such pharmaceutical dosage forms or food additives can be liquids, powders, premixes, tablets, capsules or suspensions.

Pharmaceutical amounts will generally range from about 1,000 parts per million (ppm) to about 10,000 ppm of CLA of the human's diet. However, the upper limit of the amount to be employed is not critical because CLA is nontoxic. CLA for this and other uses may also be prepared in a variety of forms. These include nontoxic sodium or potassium salts of CLA in combination with pharmaceutical diluent and active esters. CLA may also be incorporated directly into animal feed or food to be fed to a human so that CLA comprises approximately 0.01% to 2% or more by weight of the animal or human's food.

### Sequences

### 1. SEQ ID No. 1

Nucleic acid sequence of the trans-10, cis-12 conjugated linoleic acid isomerase isolated from Propionibacterium acnes (accession no. CQ766028)

### 2. SEQ ID No.2

Amino acid sequence of the trans-10, cis-12 conjugated linoleic acid isomerase isolated from Propionibacterium acnes (accession no. CQ766028)

### 3. SEQ ID No. 3

Nucleic acid sequence of the PCR-Primer ERcoPAI1

5′-AAAACTGCAGAGGAGGAAAAAAAATGGGTTCCATTTCCAAGGA-3′

### 4. SEQ ID No. 4

Nucleic acid sequence of the ERcoPAI2

5′-CGGGGTACCTCACACGAAGAACCGCGTCA-3′

### 5. SEQ ID No. 5

Nucleic acid sequence of the vector pNZ44-coPAI

### EXAMPLES

The following examples are provided in order to demonstrate and further illustrate certain preferred embodiments and aspects of the present invention and are not to be construed as limiting the scope thereof.

In the experimental disclosure which follows (and the above given description of the current invention), the following abbreviations apply: M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml (milliliters); μl (microliters); cm (centimeters); mm (millimeters); μm (micrometers); nm (nanometers); ° C. (degrees Centigrade); MQ (milli-Q water; deionized water with further volatiles removed, to provide pure water with an electrical resistance of 18.2 ohms); PSI (pounds per square inch); cDNA (copy or complimentary DNA); DNA (deoxyribonucleic acid); ssDNA (single stranded DNA); dsDNA (double stranded DNA); dNTP (deoxyribonucleotide triphosphate); RNA (ribonucleic acid); PBS (phosphate buffered saline); OD (optical density); HEPES (N-[2-Hydroxyethyl]piperazine-N-[2-ethanesulfonic acid]); HBS (HEPES buffered saline); SDS (sodium dodecylsulfate); Tris-HCl (tris[Hydroxymethyl]aminomethanehydrochloride); DMSO (dimethyl sulfoxide); EGTA (ethylene glycol-bis(β-aminoethyl ether) N,N,N′,N′-tetraacetic acid); EDTA (ethylenediaminetetracetic acid)

### Example 1

### Cultures and Media

Lactococcus lactis NZ9800 (a L. lactis NZ9700 derivative which does not produce nisin because of a deletion in the nisA gene, and contains the nisRK signal transduction genes integrated on the chromosome) was cultured at 30° C. in M17 (Difco laboratories, Detroit Mich., USA) broth and/or agar containing glucose (0.5% w/v). The probiotic strain Lactobacillus paracasei ssp. paracasei NFBC 338 (Lb. paracasei NFBC 338) was previously isolated from the human gastrointestinal tract (GIT), and obtained from University College Cork, Ireland under a restricted materials transfer agreement. Lb. paracasei NFBC 338 was routinely cultured overnight (˜17 h) in MRS broth (Oxoid Ltd., Hampshire, UK) and incubated at 37° C. under anaerobic conditions using anaerobic jars containing Anaerocult A gas packs (Merck, Darmstedt, Germany). L. lactis carrying the plasmids pNZ44 were routinely cultured in the presence of chloramphenicol (5 μg/ml) as a selective marker. Lb. paracasei NFBC harboring the vector pMSP3535 were routinely cultured with erythromycin (10 μg/ml) as selective marker. E. coli TOP 10 (Invitrogen) harbouring the plasmid pNZ44 was cultured in LB (Luria-Bertani)-media supplemented with chloramphenicol (20 μg/ml). Human colon cancer cells were obtained from the American Type Culture Collection (ATCC, Manassas, Va., USA). The t10, c12 CLA isomer (98%+purity) was obtained from Matreya (Matreya Inc., PA, USA;). Cell culture media and supplements were purchased from Sigma Aldrich Ireland Ltd. (Dublin, Ireland), unless otherwise stated. SW480 cells were maintained in Dulbecco's Minimum Essential Medium (DMEM) supplemented with 5% (v/v) fetal bovine serum, 0.2 mM L-glutamine, 1 mM HEPES and 1 unit/ml penicillin and streptomycin. SW480 cells were grown in 96 well plates and maintained at 37° C. in a humidified atmosphere and a pH of 7.2-7.4 by a required flow of 95% air and 5% CO₂.

### Example 2

### DNA Manipulation

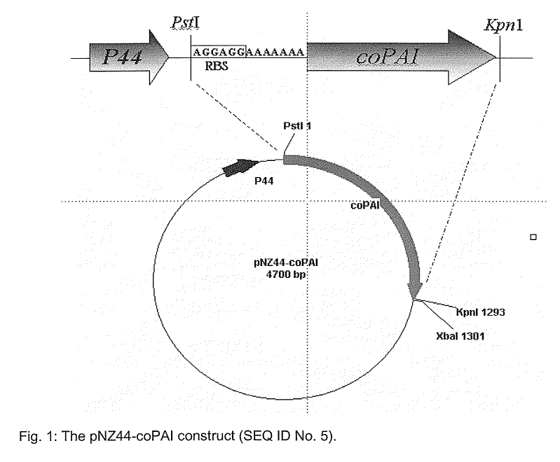

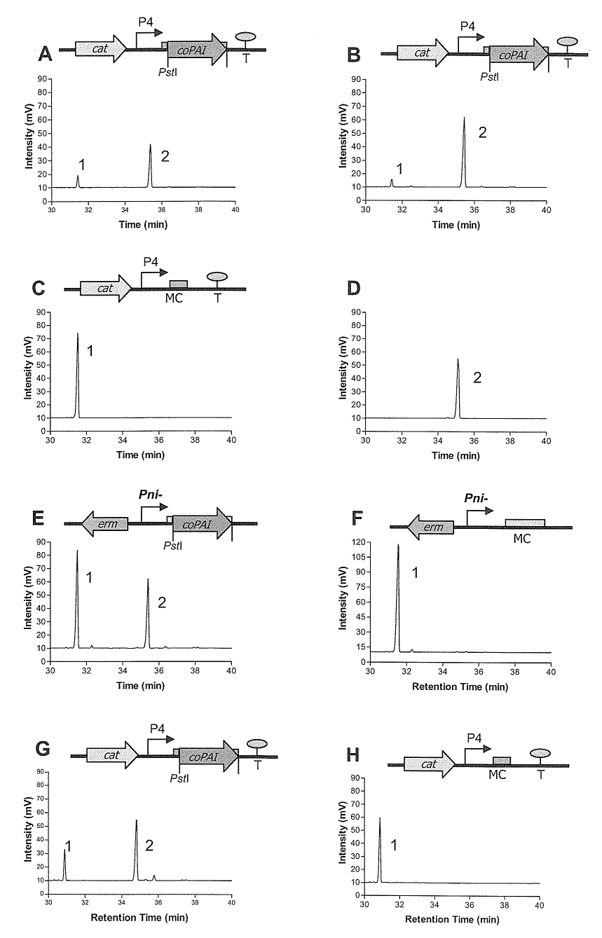

Two oligonucleotide primers were designed to amplify the complete linoleic acid isomerase (coPAI) for production of t10, c12 CLA from the original construct pC33.1-coPAI (linoleic acid isomerase gene in a plant vector; BASF, Germany). The forward primer, designated ERcoPAI1 (SEQ ID No. 3), contains a PstI restriction site and a ribosome binding site (RBS), four extra bases at the 5′ end and seven extra bases between the RBS and the gene start; 5′-AAAACTGCAGAGGAGGAAAAAAAATGGGTTCCATTTCCAAGGA-3′ (SEQ ID No. 3). The reverse primer, designated ERcoPAI2 (SEQ ID No. 4) contains a KpnI restriction site and three extra bases at the 5′ end; 5′-CGGGGTACCTCACACGAAGAACCGCGTCA-3′ (SEQ ID No.: 4). The 1278 bp coPAI gene was amplified in an Eppendorf Mastercycler Gradient (Eppendorf) with High Fidelity Expand as described by the supplier (Roche Diagnostics Limited, East Sussex, England) using 200 ng plasmid DNA (pC33.1-coPAI) as a template. PCR reactions were performed in a total volume of 50 μl containing 1 μl of each primer, 3 mM MgCl₂, 5 μl 10× Expand buffer, 1 μl dNTP's and 0.75 μl Expand DNA. PCR conditions were as follows; 10 cycles of 2 min, 15 s denaturation (94° C.), 30 s annealing (55° C.), 2 min elongation (72° C.) followed by 20 cycles of 15 s (94° C.), 30 s (55° C.), 2 min+5 s/cycle (72° C.) and finally, one 7 min cycle at 72° C. The PCR reaction mixture was analysed on a 1% (w/v) agarose gel to visualize the resulting PCR fragment. The Qiagen Plasmid Mini kit (Qiagen, West Sussex, UK) was used to isolate plasmid DNA from E. coli TOP 10, L. lactis NZ9800, and Lb. paracasei NFBC 338 with one minor modification for L. lactis and Lb. paracasei, i.e. 40 mg/ml lysozyme was added to buffer P1 and incubated for 20 min (L. lactis) and 2 hours (Lb. paracasei) at 37° C. PCR products were purified using a Qiaquick PCR Purification Kit (Qiagen). The two plasmids pNZ8048 (Nisin inducible plasmid containing PnisA promoter) and pNZ44 (a derivative of pNZ8048 in which the PnisA promoter is replaced by P44, a constitutive L. lactis chromosomal promoter) and the coPAI gene fragment were restricted with PstI and KpnI followed by ligation reaction at 15° C. with T4 DNA ligase as described by the supplier (New England Biolabs, MA USA (NEB). The construct is shown in FIG. 1. Recombinant plasmids were double digested with the same enzymes to verify the correct clone and then electroporated into L. lactis NZ9800. After confirming the correct sequence, the gene was cut out of pNZ8048-coPAI using PstI and XbaI restriction enzymes (FIG. 1) and ligated into the same sites of the Lactobacillus nisin inducible vector pMSP3535. Electrocompetent L. lactis were prepared and transformed according to the method described by de Ruyter et al., while electrocompetent Lb. paracasei NFBC 338 cells were prepared using 3.5×SMEB (1M sucrose, 3.5 mM MgCl₂) as described by Luchansky et al. Sequence analysis was performed using DNAStar software (DNAStar, Madison, Wis., USA).

### Example 3

### Screening for CLA Production

Cis-9, trans-11 and trans-10, cis-12 CLA standards were purchased from Matreya (Matreya Inc., PA, USA) and linoleic acid from Sigma (Sigma Chemical, MO, USA). The L. lactis, Lb. paracasei and E. coli clones were tested for their ability to convert free linoleic acid (0.1-0.5 mg ml⁻¹) to trans-10, cis-12 CLA as follows; 1% inoculum of an overnight culture was transferred to 10 ml broth and incubated until the culture reached OD_{600 nm} ˜0.5. Then linoleic acid (0.1-0.5 mg/ml) was added to cultures and inducible cultures were induced with 30-50 ng/ml nisin (prepared from milk solids containing 2.5% (w/v) nisin, Sigma, N-5764) followed by further incubation for 48-72 h. Cultures subjected to time experiments were grown in a larger volume of broth and 10 ml samples were taken every 12 h. Following 48-72 h incubation the culture was centrifuged and fatty acids were extracted from the supernatant and dried down under a nitrogen stream followed by methylation and analysis by gas liquid chromatography (GLC) as described (Coakley et al, 2003). All conversion rates in percentage are related to the amount of linoleic acid that was recovered and extracted from the media following incubation without culture for the same time as with culture, which represented 100% of available linoleic acid.

### Example 4

### Preparation of Fermented Oils

The E. coli pNZ44-coPAI and L. lactis pNZ44-coPAI clones were inoculated (1% overnight culture) into 500 ml of respective media and grown to OD₆₀₀=0.5 after which linoleic acid (0.5 mg/ml) was added and incubation continued for 72 hours. A linoleic acid control consisting of uninoculated media containing linoleic acid (0.5 mg/ml) was also prepared and incubated at 37° C. for 72 hours, followed by extraction of the fatty acids. Control samples prepared in triplicate from each fermentations and the unfermented linoleic acid control were also methylated and analyzed on GLC as described (Coakley et al., 2003) to calculate the ratio CLA/linoleic acid present in the sample.

### Example 5

### Anti-Proliferative Activity of Fermented Oils on Human SW480 Colon Cancer Cells

To examine the anti-proliferative activity of the oils extracted following fermentation of the cultures (L. lactis pNZ44-coPAI and E. coli pNZ44-coPAI) in respective media containing linoleic acid (0.5 mg/ml), human colon cancer cells SW480 were cultured in the presence of different concentrations of the fermented oils. Initially, 1×10⁴ cells were seeded in wells and cultured for 24 h at 37° C. to allow the cells to adhere to the surface prior to treatment with 5-20 μg t10, c12 CLA (from fermented oils and t10, c12 CLA standards in ethanol) and 5-25 μg linoleic acid (control unfermented oil and Sigma standard)/ml of media. Fermented oils from both L. lactis and E. coli, contained a mixture of linoleic acid and t10, c12 CLA at a ratio of ˜1.35:1. Control flasks were supplemented with ethanol to a final concentration of 0.1% (v/v). Following incubation for 5 days, cell viability was measured and relative cell number were determined using the MTS method (Promega Corporation, Madison, Wis., USA), a colorimetric method for determining the number of viable cells in proliferation or cytotoxicity assays subsequent to incubation with MTS tetrazolium compound. Following incubation with MTS for 2 h, the absorbance was recorded at 492 nm with a 96-well plate reader. Cell viability (%) after treatment is expressed relative to the ethanol control, which represented 100%. Three independent experiments were performed in triplicate for each treatment except for t10, c12 CLA standard (Matreya), which was performed twice in triplicate, and Student's t test was used to determine significant differences between treatments (p<0.001).

### Example 6

### Sequence Analysis

The 1278 bp gene (accession no CQ766028) from Propionibacterium acnes encodes a linoleic acid isomerase protein for t10, c12 production of 425 amino acids (SEQ ID No. 1). The molecular weight of the isomerase is 49,077 Da. Comparison with sequences in the database revealed that the cloned isomerase protein showed significant homology with proteins known as amino oxidases over most of the sequence (˜a.a 25-400; NCBI Conserved Domain Search, Marchler-Bauer et al., 2005). The isomerase showed 96% identity to a putative amino oxidase from Propionibacterium acnes (accession no Q6A8×5_PROAC; EXPASY/UniProtKB database), but only 26% identity to the next best match, a protein from the plant Oryza sativa (japonica cultivar-group, accession no Q7XR12_ORYSA; EXPASY/UniProtKB database) spanning from amino acid 145-423. The aligned region includes a flavin-binding site in these proteins. The flavin containing amine oxidase family also contains phytoene hydrogenases and related enzymes. An NAD/FAD binding domain located in the region between amino acid residue 10-39 was identified (PROSITE database). The isomerase protein is soluble and the predicted location of the protein is cytoplasmic (PSORTb, British Columbia, Canada; SOSUI, Mitaku Group, Tokyo, Japan). A putative transmembrane helic spanning from a.a. 10-26 was detected (HMMTOP; Tmpred). However, the results are rather conflicting since there was no clear consistency between the results from the different databases. No signal peptide was detected, except for Inter ProScan (European Bioinformatics Institute, Cambridge, UK) that identified a putative signal peptide between a.a 1-23.

### Example 7

### Bioconversion of Linoleic Acid

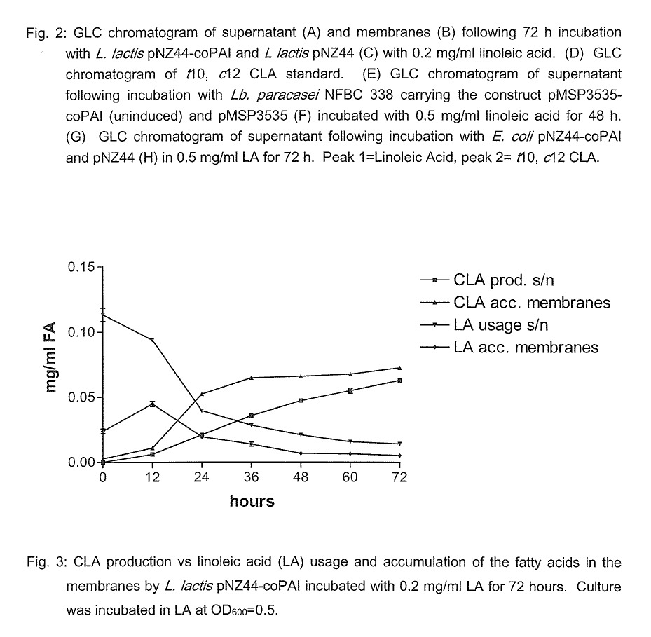

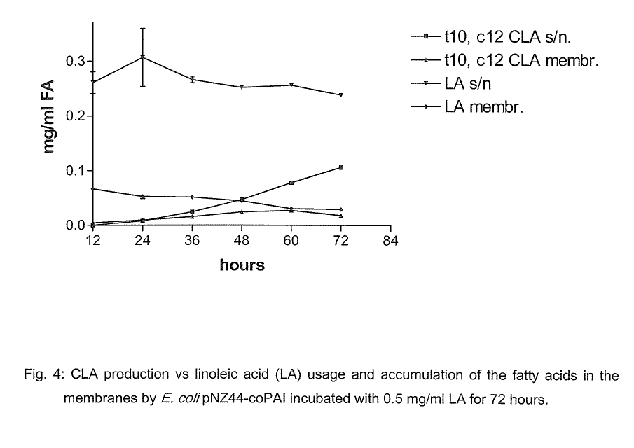

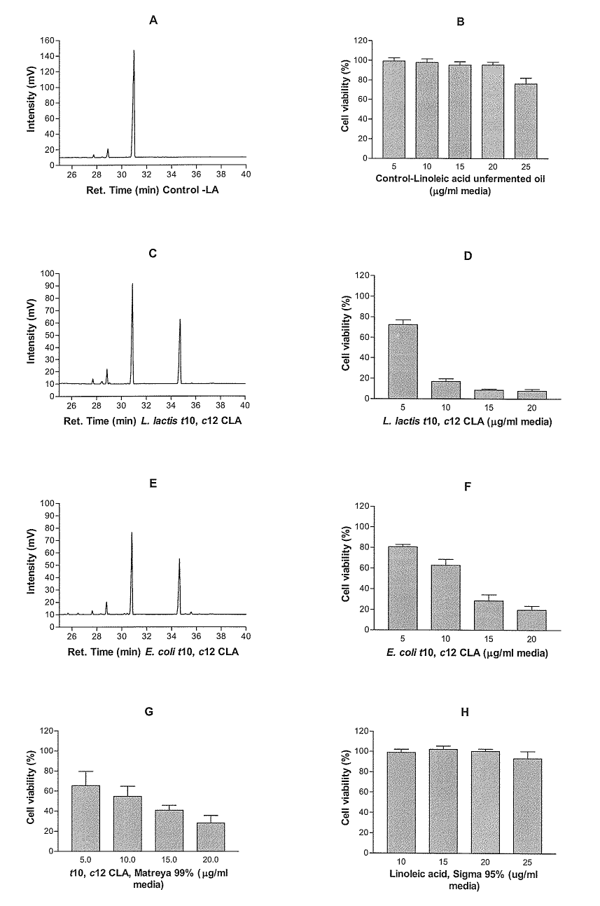

L. lactis carrying the construct pNZ44-coPAI was shown to convert free linoleic acid into t10, c12 CLA, compared with control culture L. lactis containing only the vector pNZ44, with which no conversion to CLA was detected (Table 1). L. lactis pNZ44-coPAI converted as much as >50% of the free linoleic acid to t10, c12 CLA (Table 1, FIG. 3). Given that L. lactis did not grow well if initially incubated with linoleic acid (0.4-0.5 mg/ml), the fatty acid was added when the culture was at OD₆₀₀=0.5. At this point, the culture still showed sensitivity to linoleic acid at this concentration. Greater conversion rates to CLA were observed at lower concentrations of free linoleic acid (0.1 and 0.2 mg/ml). Lb. paracasei NFBC 338 harboring the lactobacilli vector and the coPAI gene, pMSP3535-coPAI, converted nearly 30% of the LA (recovered in a control media after incubation without culture) following induction at OD₆₀₀=0.5 with 50 ng/ml nisin and incubation for 48 hours in the fatty acid (0.5 mg/ml). However, the t10, c12 CLA production by uninduced cells of Lb. paracasei NFBC 338 pMSP3535-coPAI was shown to be close to that obtained with induced cells, 24.4% compared with 28.9% from the nisin induced culture. E. coli cells carrying the construct pNZ44-coPAI converted about 40% of recovered control LA after 72 hours incubation in the presence of the fatty acid (0.5 mg/ml), whereas E. coli pNZ44 (vector control) did not produce any CLA (Table 1, FIGS. 2 and 4).

TABLE 1
% conversion of t10, c12 CLA from linoleic acid recovered in the broth.

| | | | | % conversion of t10, |
|---|---|---|---|---|
| | | Induced with/ | Amount LA added | c12 CLA from LA |
| Culture | Plasmid/construct | Uninduced | (mg/ml broth) | recovered in broth* |
| Lb. paracasei | pMSP3535-coPAI | 50 ng nisin/ml | 0.5 | 28.9 +/− 0.5 |
| NFBC338 | pMSP3535-coPAI | Uninduced | 0.5 | 24.4 +/− 0.4 |
| | pMSP3535 | 50 ng nisin/ml | 0.5 | 0 |
| | pMSP3535 | Uninduced | 0.5 | 0 |
| L. lactis NZ9800 | pNZ44-coPAI | — | 0.2 | 52.2 +/− 1.0 |
| | | | (at OD₆₀₀ = 0.5) | (+60.1 +/− 0.5 in pellet) |
| | pNZ44 | — | 0.2 | 0 |
| | | | (at OD₆₀₀ = 0.5) | |
| E. coli | pNZ44-coPAI | — | 0.5 | 39.1 +/− 1.6 |
| | pNZ44 | — | 0.5 | 0 |

*All conversion rates in percentage are related to the amount of linoleic acid that was recovered and extracted from the media following incubation without culture for the same time as with culture, which represented 100% of available linoleic acid.

### Example 8

### Isolation of Lipids from the Microorganisms

The Bifidobacterium strain was grown (2% inoculum) in 500 ml cys-MRS (0.05% (w/v) L-cysteine hydrochloride (98% pure; Sigma Chemical Co. St. Louis, Mo., USA) was added to the MRS medium) with 0.5 mg ml⁻¹ added linoleic acid (Sigma Chemical Co.) to assess bioconversion of the substrate. The linoleic acid was added as a 30 mg ml⁻¹ stock solution in distilled water containing 2% (v/v) Tween 80. The linoleic acid stock solution was previously filter-sterilised through a 0.45 mm Minisart filter and stored in the dark at −20° C. The strains were incubated anaerobically for 42 hours at 37° C. Following incubation, the fatty acids in the bacterial supernatant was extracted as follows: to 450 ml of the bacterial supernatant, 225 ml isopropanol (99% purity; Alkem Chemicals Ltd., Cork, Ireland) was added and vortexed for 30 sec. Hexane (170 ml added initially and vortex mixed before adding a further 340 ml hexane) (99% purity; LabScan Ltd., Dublin, Ireland) was added to this mixture, vortexed and centrifuged at 960×g for 5 min. The resultant supernatant (hexane layer containing lipids) was removed to a glass tube and the hexane was dried to 2-3 ml under a stream of nitrogen at 45° C. The lipids were stored under nitrogen at −20° C. Fatty acid composition of the bacterial supernatant and level of conversion of the linoleic acid to CLA was assessed following addition of an internal standard (C_{13:0} tridecanoic acid (99% pure, Sigma Chemical Co.), methylation and gas liquid chromatography (GLC), as previously described (Stanton et al., 1997).

### Example 9

### Preparation of Fatty Acid Methyl Esters (Fame) and GLC Analysis

The lipid extracts in hexane were analysed by GLC following acid-catalyzed methylation as described previously (Stanton et al., 1997).

Free fatty acids in oils such as sunflower and soybean oils were calculated as the difference between fatty acid concentrations obtained following acid and base catalyzed methylation, performed using 2 N methanolic KOH (Sigma Chemical Co.) at room temperature. The GLC was performed with reference to the internal standard C_{13:0}. Separation of the FAME was performed on a Chrompack CP Sil 88 column (Chrompack, Middleburg, The Netherlands, 100 m×0.25 mm i.d., 0.20∝m film thickness), using helium as carrier gas at a pressure of 37 psi. The injector temperature was held isothermally at 225° C. for 10 min and the detector temperature was 250_C. The column oven was held at an initial temperature of 140° C. for 8 min and then programmed at an increase of 8.5° C./min to a final temperature of 200° C., which was held for 41 min. Collected data were recorded and analyzed on a Minichrom PC system (VG Data System, Manchester, UK). The trans-10, cis-12 CLA isomer CLA isomer was identified by retention time with reference to a CLA mix (Nu-Chek-Prep. Inc., Elysian, Minn.). The percentage conversion to CLA and the remaining linoleic acid in the broth were calculated by dividing the amount of CLA and linoleic acid present in the broth after inoculation and incubation with the various cultures used with the amount of linoleic acid present in the spiked broth before incubation.

### Example 10

### Lipid Extraction of Supernatant

After transferring 10 ml of the cultures inoculated with either CLA or LA to 15 ml centrifuge tubes (Sarstedt, Numbrecht, Germany), centrifugation was performed at 2197×g for 20 min at room temperature (20° C.), using a Sanyo Mistral 2000 R centrifuge. To 4 ml of the supernatant were added 0.75 mg C 13:0 (tridecenoic acid, Sigma, 99% pure) as internal standard prior to lipid extraction, performed as follows: 2 ml isopropanol (Alkem Chemicals Ltd. Cork, Ireland, 99% purity) and 1.5 ml hexane (LabScan Ltd. Dublin, Ireland, 99% purity) were added to the supernatant and vortex mixed, and a further 3 ml of hexane were then added and the mixture, which was vortex mixed again before centrifugation at 2197×g for 5 min. All upper layer (hexane layer containing fatty acids) was transferred to a screw capped glass tube and dried down under N2 gas stream. Tubes were then stored at −20° C. prior to preparation of fatty acid methyl esters (FAME) for GLC (Gas Liquid Chromatography) analysis. Following GLC, results were calculated as mg fatty acid per ml of broth.

### Example 11

### Lipid Extraction of Pellet

After removal of supernatant, bacterial cells (pellets) from 10 ml of grown culture were washed by adding and resuspending them in 1 ml saline solution (0.137 M NaCl, 7.0 mM K₂HPO₄, 2.5 mM KH₂PO₄) and vortex mixing before centrifuging at 3632×g for 30 min. After removal of supernatant, pellets were again resuspended in 1 ml saline solution followed by centrifugation at 3632×g for 15 min and removal of the supernatant again. The cells were again resuspended in 1 ml saline solution, to which was added 0.75 mg C 13:0 (as described above for supernatant) as internal standard prior to preparation of FAME for GLC analysis. Following GLC, results were calculated as mg fatty acids from 1 ml of fully grown culture and expressed as mg fatty acids/ml.

### Example 12

### Preparation of Fatty Acid Methyl Esters (Fame)

Acid catalyzed methylation, which results in derivatisation of both free fatty acids and triglyceride bound fatty acids was performed as described below: Extracted lipids from supernatants and pellets (as described in sections 2.4.1 and 2.4.2) in screw capped glass tube, were resuspended in 12 ml, 4% methanolic HCl (v/v) (Supelco Inc. Bellefonte, Pa., USA) in methanol and vortex mixed for 10 sec. The lipids in methanolic HCl were incubated at 60° C. for 1 h with vortex mixing every 10 min. Two ml of water saturated with hexane and 5 ml of hexane were then added to the solution which was vortex mixed for 30 sec, and then allowed to stand for 30 min. The clear top layer, containing the FAME was subsequently transferred to a tube and 2 ml of water saturated with hexane were added and the solution again vortex mixed and allowed to stand for 30 min. Following this, the top layer was transferred to a new tube and the methylation reaction terminated by addition to this layer of 0.5 g anhydrous sodium sulphate (Sigma, 99% purity) and vortex mixed for 5 sec. After 1 h, the top layer was removed and stored at −20° C. prior to GLC analysis.

### Example 13

### GLC Analysis

The free fatty acids were analysed as fatty acid methyl esters (FAME) using a gas liquid chromatograph (GLC-Varian 3400, Varian, Harbor City, Calif., USA) fitted with a flame ionization detector (FID) and a Septun Programmable Injector (SPI). Quantification of fatty acids was performed with reference to the internal standard (C 13:0). Separation of fatty acids was performed on a Chrompack CP Sil 88 column (Chrompack, Middleburg, The Netherlands) (100 m×0.25 mm i.d., 0.20 m film thickness), using He as carrier gas at a pressure of 33 psi. The injector temperature was held isothermally at 225° C. for 10 min and the detector temperature was 250° C. The column oven was held at an initial temperature of 140° C. for 8 min, and then programmed at an increase of 8.5 C/min to a final temperature of 200° C., which was held for 41 min.

Collected data were recorded and analyzed on a Minichrom PC system (VG Data System, Manchester, UK). The trans-10, cis-12 CLA isomer was identified by retention time with reference to CLA standards (Matreya Inc. PA, USA), and trans-11-C18:1 and stearic acid (Sigma Chemical Co. St. Louis, Mo., USA) identified by reference to their standard fatty acids. To calculate correction factors for the CLA isomer peaks the internal standard C 13:0 was used using the following formula: Cf_{I}=(A_{IS}×Wt_{I})/(A_{I}×Wt_{IS}), where Cf_{I} is the correction factor for the actual CLA isomer, A_{IS} refers to the area of the internal standard (C 13:0), A_{I} is the area of the CLA peak, Wt_{I} is the weight of the CLA isomer and Wt_{IS} refers to the weight of the internal standard. The quantity of CLA was expressed as mg/ml broth. The response factors of the individual fatty acids were calculated relative to the area of C18:0, which was assigned a response factor of 1.00. The % conversion to CLA and the % remaining linoleic acid in the broth were calculated by dividing the amount of CLA and linoleic acid present in the broth after inoculation with the cultures used, with the amount of linoleic acid present in the spiked broth before incubation. All conversion rates in percentage are related to the amount of linoleic acid that was recovered and extracted from the media following incubation without culture for the same time as with culture, which represented 100% of available linoleic acid

### Example 14

### Anti-Proliferative Activity of Fermented Oils on Human Colon Cancer Cells SW480

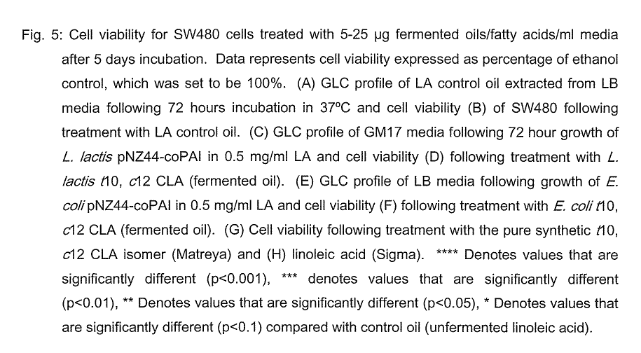

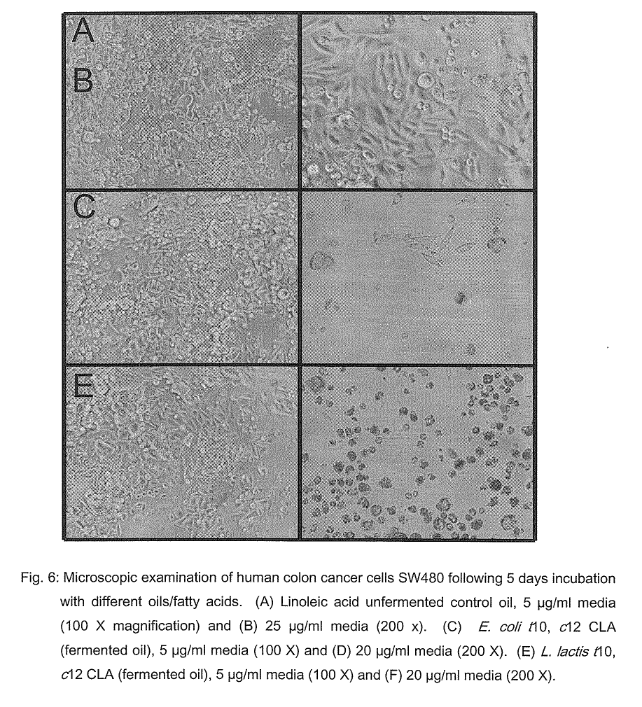

To investigate the anti-proliferative effect of oils produced following fermentation of linoleic acid by L. lactis pNZ44-coPAI and E. coli pNZ44-coPAI, human colon cancer cells SW480 were cultured in the presence of the extracted fermented oils consisting of a mixture of linoleic acid and t10, c12 CLA at a ratio of ˜1.35:1. Controls of linoleic acid extracted from LB broth after 72 hours incubation at 37° C., linoleic acid (Sigma, 95%) and the pure synthetic t10, c12 CLA isomer (Matreya) were also cultured with SW480 cells to compare the effect of the fermented oils versus the pure isomer, but also to ensure that the concentration of the added oils were below the concentration when linoleic acid starts to have a cytotoxic effect on the cancer cells. Since linoleic acid has been shown to have an anti-proliferative effect on SW480 cancer cells at 42.8 μg/ml media (152.5 μM), and a slightly proliferative effect at a concentration of 16.9 μg/ml media (60.2 μM) (Miller et al., 2003), concentrations of t10, c12 CLA (fermented oil samples) between 5-20 μg/ml media (equivalent to 6.7-27 μg of linoleic acid/ml media in the same oil sample) were chosen so as not to exceed the threshold concentration when linoleic acid inhibits cell growth. Following 5 days incubation with t10, c12 CLA between 5-20 μg/ml, there was a significant (p<0.001) reduction in growth of the SW480 cancer cells compared with control linoleic acid unfermented oil. Cell viability after treatment with 5-20 μg/ml t10, c12 CLA was reduced to 72.6%+/−13.6% (5 μg/ml)-7.9%+/−4.5% (20 μg/ml) (L. lactis CLA) and 80.7%+/−6.8% (5 μg/ml)-19.6%+/−11.8% (20 μg/ml) (E. coli CLA), compared with 99.1%+/−10.0% (5 μg/ml) −95.4%+/−7.8% (20 μg/ml) (control-unfermented LA) (FIGS. 5 and 6). Cell numbers following incubation with the highest concentration (25 μg/ml) of linoleic acid had a slightly anti-proliferative effect on the cancer cells, 76%+/−18.4% cell viability when treated with unfermented control linoleic acid and 93.2%+/−20.8% cell viability when the pure (95%) Sigma linoleic acid was used. All figures are related to ethanol controls=100% cell viability. Significant differences in cell viability was observed at all concentrations between control oil (unfermented linoleic acid) and the fermented oils (t10, c12 CLA) from L. lactis and E. coli (p<0.001). No significant difference in cell viability following treatment with control-linoleic acid (unfermented oil) and pure linoleic acid was observed. Similarly, there was no significant difference in cell viability after treatment with E. coli t10, c12 CLA (fermented oil) and the pure t10, c12 CLA (Matreya) at any concentration. However, there was a significant difference in cell viability between treatments (L. lactis t10, c12 CLA and the pure t10, c12 CLA) at concentrations 10-15 μg/ml (p<0.001) and 20 μg/ml (p<0.01).

### REFERENCES

Alonso, L., Cuesta, E. P. and Gilliland, S. E. 2003. Production of free conjugated linoleic acid by Lactobacillus acidophilus and Lactobacillus casei of human intestinal origin. J. Dairy. Sci. 86: 1941-1946.
Baumgard, L. H., Corl, B. A., Dwyer, D. A., Saebø², A. and Bauman, D. E. 2000. Identification of the conjugated linoleic acid isomer that inhibits milk fat synthesis. Am. J. Physiol. Regul. Integr. Comp. Physiol. 278: 179-184.
Belury, M. 2002. Dietary conjugated linoleic acid in health: Physiological effects and mechanisms of action. Annu. Rev. Nutr. 22: 505-531.
Blankson, H., Stakkestad, J. A., Fagertun, H., Thom, E., Wadstein, J. and Gudmundsen, O. 2000. Conjugated Linoleic Acid Reduces Body Fat Mass in Overweight and Obese Humans. J. Nutr. 130: 2943-2948.
Brown, J. M., Boysen, M. S., Jensen, S. S., Morrison, R. F., Storkson, J., Currie, R. L., Pariza, M., Mandrup, S, and McIntosh, M. K. 2003. Trans-10, cis-12 conjugated linoleic acid decreases glucose and fatty acid uptake and oxidation and inhibits PPARgamma-dependent gene expression in human preadipoicytes. J. Lipin Res. 44: 1287-1300.
Coakley, M., Ross, R. P., Nordgren, M., Fitzgerald, G., Devery, D. and Stanton, C. 2003. Conjugated linoleic acid biosynthesis by human-derived Bifidobacterium species. J. Appl. Microbiol. 94: 138-145.
Chin, S. F., Storksson, J. M., Liu, W., Albright, K. J. and Pariza, M. W. 1994. Conjugated linoleic acid (9,11- and 10,12-octa-decadienoic acid) is produced in conventional but not germ-free rats fed linoleic acid. J. Nutr. 124: 694-701.
Choi, Y., Kim, Y-C., Han, Y-B., Park, Y., Pariza M. W. and Ntambi, J. M. 2000. The trans-10,cis-12 isomer of conjugated linoleic acid downregulates Stearoyl-CoA desaturase 1 gene expression in 3T3-L1 adipocytes J. Nutr. 130: 1920-1924.
de Ruyter, P. G., Kuipers, O. P., and de Vos, W. M. 1996. Controlled gene expression systems for Lactococcus lactis with the food-grade inducer nisin. Appl Environ Microbiol. 62: 3662-3667.
Jiang, J., Björck, L. and Fonden, R. 1998. Production of conjugated linoleic acid by dairy starter cultures. J. Appl. Microbiol. 85: 95-102.
Jenkins, J. K. and Courtney, P. D. 2003. Lactobacillus growth and membrane composition in the presence of linoleic or conjugated linoleic acid. Can. J. Microbiol. 49: 51-57.
Jensen, R. G. 2002. The composition of bovine milk lipids. J. Dairy Sci. 85: 295-350.
Kankaanpää, P. E., Salminen, S. J., Isolauri E. and Lee, Y. K. 2001. The influence of polyunsaturated fatty acids on probiotic growth and adhesion. FEMS Microbiol. Lett. 194: 149-153.
Khulusi, S., Ahmed, H. A., Patel, P., Mendall, M. A. and Northfield, T. C. 1995. The effects of unsaturated fatty acids on Helicobacter pylori in vitro. J. Med. Microbiol. 42: 276-282.
Kim, Y. J., Liu, R. H., Bond, D. R. and Russell, J. B. 2000. Effect of linoleic acid concentration on conjugated linoleic acid by Butyrivibrio fibrisolvens A38. Appl. Environ. Microbiol. 66: 5226-5230.
Kim, Y. J., Liu, R. H., Rychlik, J. L. and Russell, J. B. 2002. The enrichment of a ruminal bacterium (Megasphaera elsdenii YJ-4) that produces the trans-10, cis-12 isomer of conjugated linoleic acid. J. Appl. Microbiol. 92: 976-982.
Kleerebezem, M., Beerthuyzen, M. M., Vaughan, E. E., De Vos, W. M. and Kuipers, 0.1997. Controlled gene expression systems for lactic acid bacteria: Transferable nisin-inducible expression cassettes for Lactococcus, Leuconostoc, and Lactobacillus spp. Appl. Environ. Microbiol. 63: 4581-4584.
Lee, K. N., Pariza, M. W. and Ntambi, J. M. 1998. Conjugated linoleic acid decreases hepatic stearoyl-CoA desaturase mRNA expression. Biochem Biophys Res Commun. 248: 817-821.
Luchansky, J. B., Muriana, P. M. and Klaenhammer, T. R. 1988. Application of electroporation for transfer of plasmid DNA to Lactobacillus, Lactococcus, Leuconostoc, Listeria, Pediococcus, Bacillus, Staphylococcus, Enterococcus and Propionibacterium. Mol Microbiol 2: 637-46.
Ostrowska, E., Muratitharan, M. and Cross, R. F. 1999. Dietary conjugated linoleic acids increase lean tissue and decrease fat deposition in growing pigs. J. Nutr. 129: 2037-2042.
Marchler-Bauer, A., Anderson, J. B., Cherukuri, P. F., DeWeese-Scott, C., Geer L. Y., Gwadz M., He, S., Hurwitz, D. I., Jackson, J. D, Ke, Z., Lanczyck, i C. J, Liebert C. A., Liu, C., Lu, F., Marchler, G. H, Mullokandov, M., Shoemaker, B. A, Simonyan, V., Song, J. S, Thiessen, P. A, Yamashita, R. A, Yin, J. J, Zhang, D., Bryant, S. H.2005. CDD: a Conserved Domain Database for protein classification. Nucleic Acids Res. 33: D192-6.
Miller, A., Stanton, C. and Devery, R. 2001. Modulation of arachidonic acid distribution by conjugated linoleic acid isomers and linoleic acid in MCF-7 and SW480 cancer cells. Lipids. 36: 1161-1168.
Miller, A., Stanton, C. and Devery, R. 2002. C is 9,trans 11- and trans10, cis 12-conjugated linoleic acid isomers induce apoptosis in cultured SW480 cells. Anticancer Res. 22: 3879-3888.
Miller, A., Stanton, C., Murphy, J. and Devery, R. 2003. Conjugated linoleic acid (CLA)-enriched milk fat inhibits growth and modulates CLA-responsibe biomarkers in MCF-7 and SW480 human cancer cell lines. Br. J. Nutr. 90: 877-885.
Pariza, M. W., Park, Y. and Cook, M. E. 1999. Conjugated linoleic acid and the control of obesity and cancer. Tox. Sciences. 52: 107-110.
Pariza, M. W., Park, Y. and Cook, M. E. 2000. Mechanism of action of conjugated linoleic acid: Evidence and speculation. Society for Experimental Biology and Medicine (Minireview).
Park, Y., Albright, K. J., Storkson, J. M., Liu, W. and Pariza, M. W. 1999. Evidence that the trans-10,cis-12 isomer of conjugated linoleic acid induced body composition changes in mice. Lipids. 34: 235-241.
Rosberg-Cody, E., Ross, R. P., Hussey, S., Ryan, C. A., Murphy, B. P., Fitzgerald, G. F., Devery, R. and Stanton, C. 2004. Mining the microbiota of the neonatal gastrointestinal tract for CLA-producing bifidobacteria. Appl. Environ. Micro. 70: 4635-4641.
Rosson, R. A., Deng, M., Grund, A. D. and Peng, S. S. 2001. Nucleotide encoding a propionibacterium linoleate isomerase and uses thereof. Patent WO 01/00846.
Schilling, B. and Lerch, K. 1995. Cloning, sequencing and Heterologous expression of the monoamine oxidase gene from Aspergillus niger. Mol Gen Genet. 247: 430-438.
Smedman, A. and Vessby, B. 2001. Conjugated linoleic acid supplementation in humans—metabolic effects. Lipids. 36: 773-81.
Stanton, C., Lawless, F., Kjellmer, G., Harrington, D., Devery, R., Connolly, J. F. and Murphy, J. (1997). Dietary Influences on Bovine Milk cis-9,trans-11-Conjugated Linoleic Acid Content. Journal of Food Science, 62:1083-1086.
Tavladoraki, P., Schinina, M. E., Cecconi, F., Di AgostinO, S., Manera, F., Rea, G., Mariottini, P., Federico, R. and Angelini, R. 1998. Maize polyamine oxidase: primary structure from protein and cDNA sequencing. FEBS Lett. 426: 62-66.
Thom, E., Wadstein, J. and Gudmundsen, O. b 2001. Conjugated linoleic acid reduces body fat in healthy exercising humans. J. Int. Med. Res. 2001. 29: 392-6.
van der Meer, J. R., Polman, J., Beerthuyzen, M. M., Siezen, R. J., Kuipers O. P. and De Vos, W. M. 1993. Characterization of the Lactococcus lactis nisin A operon genes nis P, encoding a subtilisin-like serine protease involved in precursor processing, and nis R, encoding a regulatory protein involved in nisin biosynthesis. J. Bacteriol. 175: 2578-2588.
Verhulst, A., Janssen, G., Parmentier, G. and Eyssen, H.1987. Isomerization of polyunsaturated long chain fatty acids by Propionibacteria. System. Appl. Microbiol. 9: 12-15.

1-20. (canceled)

21. A process for the production of trans-10, cis-12 conjugated linoleic acid in a transgenic microorganism comprising:
(a) introducing into said microorganism at least one nucleic acid molecule encoding a trans-10, cis-12 conjugated linoleic acid isomerase,
(b) culturing the transgenic microorganism obtained under (a),
(c) inducing the production of trans-10, cis-12 conjugated linoleic acid by adding linoleic acid to the culture, wherein the linoleic acid is added to a microorganism culture having an optical density (OD600) of at least 0.1,
(d) incubating the induced culture for at least 12 hours, and
(e) isolating the conjugated linoleic acid from the culture media and/or transgenic microorganism.

22. The process of claim 21, wherein the nucleic acid molecule encoding the trans-10, cis-12 conjugated linoleic acid isomerase comprises a sequence
i. as described by SEQ ID NO. 1, or
ii. having at least 50 consecutive base pairs of the sequence described by SEQ ID NO.1, or
iii. having an identity of at least 80% over a sequence of at least 100 consecutive nucleic acid base pairs to the sequence described by SEQ ID NO. 1, or
iv. hybridizing under high stringent conditions with a nucleic acid fragment of at least 50 consecutive base pairs of a nucleic acid molecule described by SEQ ID NO. 1, or
v. encoding a polypeptide having at least 75%) identity to the amino acid sequence as shown in SEQ ID NO. 2 encoding a trans-10, cis-12 conjugated linoleic acid isomerase.

23. The process of claim 22, wherein the nucleic acid molecule encoding said trans-10, cis-12 conjugated linoleic acid isomerase is isolated from a rumen bacterium.

24. The process of claim 23, wherein the trans-10, cis-12 conjugated linoleic acid isomerase is isolated from Megashera elsdenii.

25. The process of claim 23, wherein the nucleic acid molecule encoding said trans-10, cis-12 conjugated linoleic acid isomerase is isolated from a microorganism belonging to the genus Propionibacterium.

26. The process of claim 25, wherein the trans-10, cis-12 conjugated linoleic acid isomerase is isolated from Propionibacterium acnes.

27. The process of claim 21, wherein the microorganism used in step (a) belongs to the family selected from the group consisting of Lactobacillaceae, Streptococcaceae, Propionibacteriaceae, Enterobacteriaceae, and Bifidobacteriaceae.

28. The process of claim 27, wherein the transgenic microorganism belongs to the genus selected from the group consisting of Lactococcus, Lactobacillus, Propionibacterium, Escherichia, and Bifidobacterium.

29. The process of claim 28, wherein the transgenic microorganism belongs to the group consisting of the species Lactococcus lactis, Lactobacillus paracasei, and Escherichia coli.

30. The process of claim 21, wherein linoleic acid is converted at a bioconversion rate of higher than 10%.

31. A process for the production of feed or food products enriched in the conjugated linoleic acid comprising adding the conjugated linoleic acid produced in claim 21 in the production of feed or food products.

32. A process for the production of nutraceuticals enriched in the conjugated linoleic acid comprising adding the conjugated linoleic acid produced in claim 21 in the production of nutraceuticals.

33. A food or feed-product or a neutraceutical enriched in conjugated linoleic acid, comprising the conjugated linoleic acid produced by the process of claim 21.

34. A transgenic microorganism expressing a nucleic acid molecule encoding a trans-10, cis-12 conjugated linoleic acid isomerase, wherein said nucleic acid molecule is functionally linked to at least one heterologous promoter sequence and wherein said nucleic acid molecule comprises
i. the nucleic acid sequence as described by SEQ ID NO: 1, or
ii. a nucleic acid sequence having at least 50 consecutive base pairs of the sequence described by SEQ ID NO: 1, or
iii. a nucleic acid sequence having an identity of at least 80% over a sequence of at least 100 consecutive nucleic acid base pairs to the sequence described by SEQ ID NO: 1, or
iv. a nucleic acid sequence hybridizing under high stringent conditions with a nucleic acid fragment of at least 50 consecutive base pairs of a nucleic acid molecule described by SEQ ID NO: 1, or
v. a nucleic acid sequence encoding a polypeptide having at least 75% identity to the amino acid sequence as shown in SEQ ID NO. 2 encoding a trans-10, cis-12 conjugated linoleic acid isomerase.

35. A process for producing a probiotic in food or feed, comprising utilizing the transgenic microorganism of claim 34 as a probiotic in food or feed.

36. The process of claim 35, wherein the microorganism belongs to the genus selected from the group consisting of Lactococcus, Lactobacillus, Propionibacterium, Escherichia, and Bifidobacterium.

37. The process of claim 36, wherein the microorganism is selected from the group consisting of Bifidobacterium breve, Bifidobacterium dentium, and Bifidobacterium pseudocatenulatum.

38. A fermented oil comprising the conjugated linoleic acid produced by the process of claim 21.

39. A method for the production of a medicament or therapeutic agent for the treatment of cancer, comprising producing a medicament or therapeutic agent comprising the fermented oil of claim 38.

40. A method for the treatment of colon cancer, comprising administering the medicament or therapeutic agent produced by the process of claim 39.


SEQUENCE LISTING (5 sequences)

SEQ ID NO: 1 - DNA, 1278 bp, Propionibacterium acnes
  CDS (1)..(1275)
atgggttccatttccaaggactccagaattgctattattggtgctggcccggccgggctggctgccggaatgtacctcgaacaggccggatttcacgactacacgatcctggaacgcaccgaccacgtcggaggcaagtgccactcaccgaactaccacggccgtcgttatgagatgggggccatcatgggcgtccccagttacgacaccatccaggagatcatggatcgcactggcgacaaggtcgacgggccgaaactgcgtcgcgagttcctgcacgaggacggcgagatctacgtcccggaaaaggatccagtgcgtggtccgcaggtcatggcagcagtgcagaagctgggccagttgctcgcgacgaagtaccagggatatgacgccaacggccactacaacaaggttcacgaggacctcatgctgcccttcgacgagttcctcgccctcaacgggtgcgaggccgcccgagacctgtggatcaaccccttcacggccttcggctacgggcacttcgacaacgtcccggccgcctacgtgctgaagtacctcgacttcgtcaccatgatgtcctttgccaagggagatctgtggacgtgggccgacggcacccaggcgatgttcgagcacctcaacgccaccctggagcacccggccgaacgcaacgttgacatcactcgcatcacccgcgaggacggcaaggtccacattcacaccacggactgggatcgcgagtccgacgtcctcgtcctcaccgtcccgctggaaaagttcctcgactactccgacgcggacgatgacgagcgggagtacttctcgaagatcatccaccagcagtacatggtggatgcctgcctggtgaaggagtacccgaccatctccgggtacgtccccgacaacatgaggcccgaacgtctcgggcacgtcatggtttactaccaccgctgggctgatgatccgcaccagatcatcacgacctacctgctacgtaaccatccggactacgcggacaagactcaggaggagtgccgccagatggtcctcgacgacatggagaccttcggtcatccggtcgagaagatcatcgaggagcagacctggtactacttcccgcacgttagctcggaggactacaaggccgggtggtacgagaaggtcgagggaatgcagggtcgtcgcaacaccttctacgccggagaaattatgagtttcggtaatttcgacgaggtgtgccactactcgaaggacctggtgacgcggttcttcgtgtga

SEQ ID NO: 2 - protein, 425 aa, Propionibacterium acnes
MGSISKDSRIAIIGAGPAGLAAGMYLEQAGFHDYTILERTDHVGGKCHSPNYHGRRYEMGAIMGVPSYDTIQEIMDRTGDKVDGPKLRREFLHEDGEIYVPEKDPVRGPQVMAAVQKLGQLLATKYQGYDANGHYNKVHEDLMLPFDEFLALNGCEAARDLWINPFTAFGYGHFDNVPAAYVLKYLDFVTMMSFAKGDLWTWADGTQAMFEHLNATLEHPAERNVDITRITREDGKVHIHTTDWDRESDVLVLTVPLEKFLDYSDADDDEREYFSKIIHQQYMVDACLVKEYPTISGYVPDNMRPERLGHVMVYYHRWADDPHQIITTYLLRNHPDYADKTQEECRQMVLDDMETFGHPVEKIIEEQTWYYFPHVSSEDYKAGWYEKVEGMQGRRNTFYAGEIMSFGNFDEVCHYSKDLVTRFFV

SEQ ID NO: 3 - DNA, 43 bp, Artificial Sequence
  primer sequence
aaaactgcagaggaggaaaaaaaatgggttccatttccaagga

SEQ ID NO: 4 - DNA, 29 bp, Artificial Sequence
  primer sequence
cggggtacctcacacgaagaaccgcgtca

SEQ ID NO: 5 - DNA, 3394 bp, Artificial Sequence
  vector
taacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatgaatcgtgatgtgtgagggaaaggagtcgcttttatggccaaaccatgggtactgcaggcatgcggtaccactagttctagagagctcaagctttctttgaaccaaaattagaaaaccaaggcttgaaacgttcaattgaaatggcaattaaacaaattacagcacgtgttgctttgattgatagccaaaaagcagcagttgataaagcaattactgatattgctgaaaaattgtaatttataaataaaaatcaccttttagaggtggtttttttatttataaattattcgtttgatttcgctttcgatagaacaatcaaagcgagaataaggaagataaatcccataagggcgggagcagaatgtccgagactaatgtaaatttgtccaccaattaaaggaccgataacgcgagcttctcgagtgcatattttcggcaatcttctcaatgagatgctcttcagcatgttcaatgatgtcgattttttattaaaacgtctcaaaatcgtttctgagacgttttagcgtttatttcgtttagttatcggcataatcgttaaaacaggcgttatcgtagcgtaaaagcccttgagcgtagcgtgctttgcagcgaagatgttgtctgttagattatgaaagccgatgactgaatgaaataataagcgcagcgtccttctatttcggttggaggaggctcaagggagtttgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttgaggggtttttcaatcgccaacgaatcgccaacgttttcgccaacgttttttataaatctatatttaagtagctttattgttgtttttatgattacaaagtgatacactaattttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaatatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagcccccctgacgaaagtcgacggcaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccttggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaagaaggtttttatattacagctccaagatc